SEQUENCE LISTING (29 sequences)

SEQ ID NO: 1 - protein, 5 aa, Homo sapiens
FVFLM

SEQ ID NO: 2 - protein, 5 aa, Homo sapiens
FVFAM

SEQ ID NO: 3 - protein, 5 aa, Homo sapiens
FVALM

SEQ ID NO: 4 - protein, 5 aa, Homo sapiens
FVFLA

SEQ ID NO: 5 - protein, 5 aa, Homo sapiens
FLVFI

SEQ ID NO: 6 - protein, 5 aa, Homo sapiens
FLMII

SEQ ID NO: 7 - protein, 5 aa, Homo sapiens
FLFVL

SEQ ID NO: 8 - protein, 5 aa, Homo sapiens
FLFVV

SEQ ID NO: 9 - protein, 5 aa, Homo sapiens
FLFLI

SEQ ID NO: 10 - protein, 6 aa, Homo sapiens
FLFFIL

SEQ ID NO: 11 - protein, 5 aa, Homo sapiens
FLMFI

SEQ ID NO: 12 - protein, 5 aa, Homo sapiens
FMLLI

SEQ ID NO: 13 - protein, 6 aa, Homo sapiens
FIIMIL

SEQ ID NO: 14 - protein, 5 aa, Homo sapiens
FLFCI

SEQ ID NO: 15 - protein, 5 aa, Homo sapiens
FLFAV

SEQ ID NO: 16 - protein, 5 aa, Homo sapiens
FVYLI

SEQ ID NO: 17 - protein, 5 aa, Homo sapiens
FAFLM

SEQ ID NO: 18 - protein, 5 aa, Homo sapiens
AVFLM

SEQ ID NO: 19 - protein, 5 aa, Homo sapiens
FCICV

SEQ ID NO: 20 - protein, 5 aa, Homo sapiens
FCVCF

SEQ ID NO: 21 - protein, 5 aa, Homo sapiens
FIVCV

SEQ ID NO: 22 - protein, 5 aa, Homo sapiens
FCVGV

SEQ ID NO: 23 - protein, 5 aa, Homo sapiens
FCVLV

SEQ ID NO: 24 - protein, 5 aa, Homo sapiens
FLVGV

SEQ ID NO: 25 - protein, 5 aa, Homo sapiens
FSVSV

SEQ ID NO: 26 - protein, 5 aa, Homo sapiens
FSVCV

SEQ ID NO: 27 - protein, 5 aa, Homo sapiens
FVCVG

SEQ ID NO: 28 - protein, 414 aa, Homo sapiens
MPSSVSWGILLLAGLCCLVPVSLAEDPQGDAAQKTDTSHHDQDHPTFNKITPNLAEFAFSLYRQLASTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 29 - protein, 8 aa, Homo sapiens
RKRRFLGF

(19) United States
(12) Patent Application Publication
(10) Pub. No.: US 2008/0051330 A1
(43) Pub. Date: Feb. 28, 2008

(54) Inhibitors of serine protease activity, methods and compositions for treatment of herpes viruses

(76) Inventor: Leland Shapiro, Denver, CO (US)

Correspondence Address: DON D. CHA, 17225 W. 12TH AVE., GOLDEN, CO, 80401, US

(21) Appl. No.: 11/404,041

(22) Filed: Apr. 14, 2006

Related U.S. Application Data
(63) Continuation of application No. 09/518,076, filed on Mar. 3, 2000.
(60) Provisional application No. 60/123,167, filed on Mar. 5, 1999.
(60) Provisional application No. 60/153,942, filed on Sep. 15, 1999.

(51) Int. Cl. A61K 38/16; A61K 31/10; A61K 31/165; A61K 31/18; A61K 31/195; A61K 31/21; A61K 31/41; A61K 31/4192; A61K 31/4196; A61K 31/4245; A61K 31/426; A61K 31/433; A61K 31/4427; A61K 31/47; A61K 31/496; A61K 31/498; A61K 31/506; A61K 31/5377; A61P 31/22; A61K 31/55; A61K 31/551; A61K 38/08

(52) U.S. Cl. 514/12; 514/17; 514/217.1; 514/218; 514/236.2; 514/254.03; 514/256; 514/266.23; 514/314; 514/340; 514/361; 514/364; 514/369; 514/381; 514/383; 514/506; 514/565; 514/600; 514/602; 514/637; 514/706; 514/708; 514/789

(57) ABSTRACT

Novel compositions and methods of treating and preventing a viral infection are provided. A method of blocking a viral infection facilitated by a serine proteolytic (SP) activity is disclosed, which involves administering to a subject suffering or about to suffer from a viral infection a therapeutically effective amount of a substance having serine protease inhibitory activity or serpin activity. Among the substances found to be useful are α₁-antitrypsin (AAT), peptide derivatives from the carboxy terminal end of AAT and synthetic drugs mimicking the action of such substances. The invention is particularly well suited for checking a viral infection mediated by members of herpesviridae family.

### 1. FIELD OF THE INVENTION

In general, the present invention relates to enzyme inhibitors and their respective ligands. More particularly, the present invention relates to substances exhibiting inhibitory activity toward viral replication and spread, which are facilitated by serine protease activity. The inhibitory compounds comprise naturally occurring and man-made serine protease inhibitors and molecules exhibiting alpha-1-antitrypsin activity.

### 2. BACKGROUND OF THE INVENTION

Serine proteases serve an important role in human physiology by mediating the activation of vital functions. In addition to their normal physiological function, serine proteases have been implicated in a number of pathological conditions in humans. Serine proteases are characterized by a catalytic triad consisting of aspartic acid, histidine and serine at the active site.

The naturally occurring serine protease inhibitors are usually, but not always, polypeptides and proteins which have been classified into families primarily on the basis of the disulfide bonding pattern and the sequence homology of the reactive site. Serine protease inhibitors, such as serpins, have been found in microbes, as well as in the tissues and fluids of plants, animals, insects and other organisms. Protease inhibitor activities were first discovered in human plasma by Fermi and Pernossi in 1894. At least nine separate, well-characterized proteins are now identified, which share the ability to inhibit the activity of various proteases. Several of the inhibitors have been grouped together, namely alpha-1-proteinase inhibitor, antithrombin III, antichymotrypsin, C1-inhibitor, and alpha-2-antiplasmin, which are directed against various serine proteases, i.e., leukocyte elastase, thrombin, cathepsin G, chymotrypsin, plasminogen activators, and plasmin. These are referred to as the alpha-1-proteinase inhibitor class. The protein alpha-2-macroglobulin inhibits members of all four catalytic classes: serine, cysteine, aspartic, and metalloproteases. However, other types of protease inhibitors are class specific. The alpha-1-proteinase inhibitor (also known as α₁-antitrypsin or AAT) and inter-alpha-trypsin inhibitor inhibit only serine proteases, alpha-1-cysteine protease inhibitor inhibits cysteine proteases, and alpha-1-anticollagenase inhibits collagenolytic enzymes of the metalloenzyme class.

Human neutrophil elastase (NE) is a proteolytic enzyme secreted by polymorphonuclear leukocytes in response to a variety of inflammatory stimuli. The degradative capacity of NE, under normal circumstances, is modulated by relatively high plasma concentrations of α₁-antitrypsin (AAT). However, stimulated neutrophils produce a burst of active oxygen metabolites, some of which (hypochlorous acid for example) are capable of oxidizing a critical methionine residue in AAT. Oxidized AAT has been shown to have a limited potency as a NE inhibitor and it has been proposed that alteration of this protease/antiprotease balance permit NE to perform its degradative functions in localized and controlled environments.

Alpha-lb 1-proteinase inhibitor also known as alpha-1-antitrypsin (α₁-antitrypsin or AAT) is a glycoprotein of MW 51,000 with 394 amino acids and 3 oligosaccharide side chains. Human AAT was named anti-trypsin because of its initially discovered ability to inactivate pancreatic trypsin. Human AAT is a single polypeptide chain with no internal disulfide bonds and only a single cysteine residue normally intermolecularly disulfide-linked to either cysteine or glutathione. The reactive site at position 358 of AT contains a methionine residue, which is labile to oxidation upon exposure to tobacco smoke or other oxidizing pollutants. Such oxidation can reduce the biological activity of AT; therefore substitution of another amino acid at that position, i.e. alanine, valine, glycine, phenylalanine, arginine or lysine, produces a form of AT which is more stable. AAT can be represented by the following formula:

MPSSVSWGILLLAGLCCLVPVSLAEDPQGDAAQKTDTSHHDQDHPTFNKI TPNLAEFAFSLYRQLASTNIEFFSPVSIATAFAMLSLGTKADTHDEILEGLNFNLTEI PEAQIHEGFQELLRTLNQPDSQLQLTTGNGLFLSEGLKLVDKFLEDVKKLYHSEA FTVNFGDTEEAKKQINDYVEKGTQGKIVDLVKELDRDTVFALVNYIFFKGKWER PFEVKDTEEEDFHVDQVTTVKVPMMKRLGMFNIQHCKKLSSWVLLMKYLGNA TAIWFFLPDEGKLQHLENELTHDIITKFLENEDRRSASLHLPKLSITGTYDLKSVLGQ LGITKVFSNGADLSGVTEEAPLKLSKAVHKAVLTIDEKGTEAAGAMFLEAIPMSI PPEVKFNKPFVFLMIEQNTKSPLFMGKVVNPTQK. (Details of the sequence can be found for example in U.S. Pat. No. 5,470,970 incorporated herein by reference in its entirety).

The C-termini of human antitrypsin (AAT), is homologous to antithrombin (ATIII), antichymotrypsin (ACT), C1-inhibitor, tPA-inhibitor, mouse AT, mouse contrapsin, barley protein Z, and ovalbumin. Its normal plasma concentration ranges from 1.5 to 3.5 mg/ml although it can behave as an acute phase reactant by increasing 3-4-fold during host response to inflammation and/or tissue injury such as with pregnancy, acute infection, and tumors. It easily diffuses into tissue spaces and forms a 1:1 complex with a target protease, principally neutrophil elastase. Other enzymes such as trypsin, chymotrypsin, cathepsin G, plasmin, thrombin, tissue kallikrein, and factor Xa can also serve as substrates. The enzyme/inhibitor complex is then removed from circulation by binding to serpin-enzyme complex (SEC) receptor and catabolized by the liver and spleen cells. Humans with circulating levels of AAT less than 15 percent (%) of normal are susceptible to the development of lung disease, e.g., familial emphysema, at an early age. Therefore, it appears that this inhibitor represents an important part of the defense mechanism against attack by serine proteases.

It is known that in some instances the degradative action of serine proteases results in serious pathological conditions or disease states. For example, elastase is a protease which causes degradation and fragmentation of elastic fibers as a result of its proteolytic activity on rubber-like elastin. Other connective tissue proteins, such as type I, III, and IV collagens, the protein portion of proteoglycans, and laminin can be also cleaved by elastase. Tissues comprising the lungs, bronchi, ear, and skin contain large amounts of elastin. Excessive degradation of elastin has been also associated with arthritis, atherosclerosis, certain skin diseases, pulmonary emphysema and adult respiratory-distress syndrome. Therefore, by inhibiting the activity of elastase it is possible to treat a wide variety of pathological conditions including pulmonary emphysema, various clotting disorders and inflammatory processes.

One illustration of the importance of the catalytic activity of serine proteases is provided by the role of human neutrophil elastase and one of its natural inhibitors, AAT in the pathogenesis of emphysema or cystic fibrosis. In the lungs of healthy individuals there is a balance between the levels of elastase and its inhibitors. The elastase serves in the repair and turnover of connective tissues (elastin) and the AAT is involved in the regulation and clearance of elastase. Disruption of the elastase/AAT balance leads to increased elastin degradation and, hence, to elastic tissue destruction. A prolonged imbalance leads to an irreversible dilation of pulmonary airways and damage to the respiratory tissues of the lung, a condition known as pulmonary emphysema. As another example, oxidants from the condensate of cigarette smoke have been shown to drastically reduce the elastase binding affinity of AAT by oxidizing a methionine residue within the reactive site. A final example involves both elevated levels of elastase and simultaneously lower levels of functional AAT inhibitor. The inflammatory response to foreign particulate matter or cigarette smoke leads to elevated levels of polymorphonuclear leukocytes in the lungs. These cells disrupt the protease/protease inhibitor balance by secretion of proteolytic enzymes, e.g., elastase. They also secrete oxidants including myeloperoxidase which appear to oxidatively inactive AAT.

So far, AAT is one of few naturally-occurring mammalian serine protease inhibitors clinically approved for the therapy of protease imbalance. Therapeutic AAT became commercially available since the mid 1980s and are prepared by various purification methods (see for example Bollen et al., U.S. Pat. No. 4,629,567; Thompson et al., U.S. Pat. No. 4,760,130; U.S. Pat. No. 5,616,693; WO 98/56821). PROLASTIN is a trademark for a purified variant of AAT and is currently sold by Bayer Company (U.S. Pat. No. 5,610,285 Lebing et al., Mar. 11, 1997). Recombinant unmodified and mutant variants of AAT produced by genetic engineering methods are also known (U.S. Pat. No. 4,711,848); methods of use are also known, e.g., AAT gene therapy/delivery (U.S. Pat No. 5,399,346 to French Anderson et al.).

Numerous serine protease inhibitors have been identified. These include transition state analog peptides such as decanoyl-Arg-Lys-Arg-Arg-psi [CH2NH]-Phe-Leu-Gly-Phe-NH2, substrate analogues such as decanoyl-RVKR-chloromethylketone, suicide substrates such as diisopropyl fluorophosphate (DFP), microbial inhibitors like leupeptin and antipain, trypsin-type protease inhibitors such as aprotinin, HI-30, E-64, trypstatin, bikunin, H130, N-alpha-tosyl-L-lysyl-chloromethyl ketone, and aryl guanidinobenzoates. Other small protease inhibitory molecules (man-made molecules) such as disclosed in U.S. Pat. Nos. 5,891,852; 5,874,585; 5,869,455; 5,863,899; 5,861,380; 5,849,863; 5,843,900; 5,834,431; 5,811,241; 5,807,829; 5,801,148; 5,750,506; 5,700,779; 5,663,416; 5,635,593; 5,618,792; 5,610,140; 5,416,191; 5,314,910; 5,281,617; 5,240,956; 5,216,022; 5,214,191 as well as PCT publications WO 98/49190; WO 98/24806; WO 98/06417; WO 97/10222; WO 97/09347; and WO 97/09346 are known and the content of these patents and PCT publications is incorporated in their entirety by way of reference.

Yet, despite all these efforts not a single compound has been considered clinically acceptable. This is mainly due to the fact that serine protease inhibitors in general have a broad inhibitory range not only toward HIV facilitating enzymes but also against vital proteolytic enzymes that are necessary for a normal function of a host.

2.1 AAT and Herpes Virus Infections

Herpes viruses are double stranded DNA viruses that replicate in host cell nuclei. The herpes virion is constituted from over 30 different proteins, which are assembled within the host cell. About 6-8 are used in the capsid. The preferred host cells for herpes viruses are vertebrate cells. The herpes viruses are animal viruses of significant clinical importance as they are the causative agents of many diseases. Epstein-Barr virus has been implicated in cancer initiation; cytomegalovirus (CMV) is the greatest infectious threat to AIDS patients; and Varicella Zoster Virus, is a causative agent of chicken pox and shingles. Herpes simplex virus subtypes 1 and 2 (HSV-1, HSV-2), are herpes viruses that are among the most common infectious agents encountered by humans. These viruses cause a broad spectrum of diseases, which range from relatively insignificant infections such as recurrent herpes simplex labialis, to severe and life-threatening diseases such as herpes simplex encephalitis. A large percentage of the United States population is affected by some form of a herpes virus infection. An estimated 98 million persons suffer each year from herpes labialis (HSV-1) and about 30 million cases of genital herpes (HSV-2) are recorded each year. Commonly these viruses are transmitted by virus exposure at mucosal surfaces and abraded skin, permitting the entry of virus and viral replication in the epidermis and dermis. In addition to clinically apparent lesions, latent infections can persist, particularly in nerve cells. This is a difficult infection to eradicate. This scourge has largely gone unchecked due to the inadequacies of available treatment.

The vast majority of the human experience with these infections is associated with rather benign symptoms, such as malaise, fever, chills, rhinitis and diarrhea. However, herpes viruses are implicated in more serious health problems such as soft tissue sarcoma, carcinoma, metastatic disease, plasmacytoma, myeloma, lymphoma, certain heritable states including retinoblastoma, Li-Fraumeni syndrome, Gardner's syndrome, Werner's syndrome, nervoid basal cell carcinoma syndrome, neurofibromatosis type 1, and some iminunodeficiency syndromes. Other conditions of notable clinical interest are leukoplakia, vesiculoulcerative mucosal diseases, idiopathic burning mouth, aphthous ulceration

For example, a single species of herpes family viruses, i.e., Epstein Barr virus (EBV) is associated with endemic Burkitt's lymphoma, acquired immune deficiency syndrome (AIDS)-related lymphoma, post-transplantation lymphoproliferative disease, Hodgkin's disease (HD), and rare T-cell lymphomas. Epstein-Barr virus is also associated with oral hairy leukoplakia, lymphoproliferative disease, lymphoepithelial carcinoma, B-cell lymphomas, and non-keratinising and squamous cell nasopharyngeal carcinoma.

Human herpesvirus-8 has been implicated in all forms of Kaposi's sarcoma, primary effusion lymphomas, multiple myeloma, angioimmunoblastic lymphadenopathy, and Castleman's disease. HHV-8 is also associated with certain lymphomas including rare B cell lymphomas called body-cavity-based lymphomas, epithelial tumors in kidney transplant recipients, malignant mesothelioma, angiosarcoma, and angiolymphoid hyperplasia.

Human herpesvirus-6 has been detected in and associated with lymphoproliferative disease, lymphomas, Hodgkin's disease, and oral squamous cell carcinoma.

Primary infection with HSV-1 rarely causes significant problems although widespread involvement in atopic eczema can be life-threatening as can associated encephalitis. Keratoconjunctivitis, pharyngitis and hepatitis can also complicate primary infection. Twenty to forty percent of the population at some stage have recurrent orolabial infections with HSV although in only one percent of these cases is this recurrence severe. Recurrent erythema multiforme appears to be associated with HSV-1 as sixty five percent of patients are thought to have preceding herpes labialis.

Herpes zoster infection can cause polyneuropathies, motor neuropathies, sensory neuronopathies, polyradiculoneuropathies, autonomic neuropathies, focal or multifocal cranial neuropathies, radiculopathies, and plexopathies, typically resulting from tumor infiltration.

People with acquired immunodeficiency syndrome (AIDS) are at an increased risk of Kaposi's sarcoma, non-Hodgkin's lymphoma, Hodgkin's disease, squamous cell carcinoma of the conjunctiva, and childhood leiomyosarcoma. It is striking that most of these cancers have been associated with specific human herpesvirus (HHV) infections: HHV-8 with Kaposi's sarcoma and the closely related Epstein-Barr virus with non-Hodgkin's lymphoma, Hodgkin's disease, and possibly also with childhood leiomyosarcoma. Moreover, similar associations between these viruses and cancer have been found, albeit inconsistently, in people who are not immunosuppressed. A general review on some aspects of herpesviridae-related diseases can be found in Flaitz and Hicks (Flaitz C M, Hicks M J. Molecular piracy: the viral link to carcinogenesis. Oral Oncol 1998 November; 34 (6):448-53).

Despite some successful therapy by a variety of nucleoside analogues additional and improved treatments directed against other viral targets are desperately needed. Protease activity appears to be essential for viral replication within the entire group of herpes viruses. Thus, it would be desirable to characterize the herpes group proteases as potential antiviral targets. Some of herpes proteases have been purified and described see, e.g., U.S. Pat. No. 5,478,727 to Roizman et al., and U.S. Pat. No. 5,486,470 to Darke et al., incorporated herein by way of reference. DiIanni et al., have first demonstrated that HSV-1 protease is a serine protease responsible for proteolytic processing of the virus assembly protein, ICP35 (infected cell protein 35). (Dilanni C L, Drier D A, Deckman I C, McCann P J 3d, Liu F, Roizman B, Colonno R J, Cordingley M G. Identification of the herpes simplex virus-1 protease cleavage sites by direct sequence analysis of autoproteolytic cleavage products. Biol Chem 1993 January 25;268(3):2048-51).

Early inhibitor studies indicated that the HSV-1 protease is sensitive to the serine protease inactivator diisopropyl fluorophosphate—a compound which is fatal to humans (DiIanni C L, Stevens J T, Bolgar M, O'Boyle D R 2nd, Weinheimer S P, Colonno R J. Identification of the serine residue at the active site of the herpes simplex virus type 1 protease. J Biol Chem 1994 April 29;269(17):12672-6). Further search revealed proteinase inhibitor aprotinin, which after digestion with clostripain, a cysteine proteinase, yielded five oligopeptide fragments. Two fragments exhibited both antiviral and antibacterial activities, two fragments only antiviral activity, and one fragment showed no antimicrobial activity. However, antivirally active oligopeptides were devoid of trypsin inhibiting activity meaning that trypsin inhibitors would be not effective against HSV (Pellegrini A, Thomas U, Franchini M, Stockli M, Klauser S, Hunziker P, von Fellenberg R. Identification of an aprotinin antiviral domain. FEBS Lett 1994 May 16;344(2-3):261-5).

Cystatin C is a human cysteine proteinase inhibitor present in extracellular fluids. Cystatin C and a tripeptide derivative (Z-LVG-CHN2) that mimics its proteinase-binding center, display strong inhibitory effects on HSV replication at molar concentration lower than that of acyclovir, the drug currently most used against HSV infections (Bjorck L, Grubb A, Kjellen L. Cystatin C, a human proteinase inhibitor, blocks replication of herpes simplex virus. J Virol 1990 February; 64(2):941-3). Oryzacystatin (OC) is a plant cystatin originating from rice seed and was investigated on the replication of herpes simplex virus type I (HSV-1) in vitro and in vivo. The effect of OC was comparable to that of acyclovir, indicating that thiol proteinases are important for replication process of HSV-1 (Aoki H, Akaike T, Abe K, Kuroda M, Arai S, Okamura R, Negi A, Maeda H. Antiviral effect of oryzacystatin, a proteinase inhibitor in rice, against herpes simplex virus type 1 in vitro and in vivo. Antimicrob Agents Chemother 1995 April; 39(4):846-9).

Thus, prior to the present invention disclosed herewith, it was believed that conventional serine protease inhibitors are not promising drug candidates targeting herpes proteases and therefore the search for more suitable inhibitors led to cysteine protease antagonists, which appeared to be more effective. As a result no suitable approach has been adopted and novel ideas are being sought to provide better anti-herpes drugs (Holwerda BC. Herpesvirus proteases: targets for novel antiviral drugs. Antiviral Res 1997 June; 35(1):1-21).

In fact, even this consensus is far from being unanimous as some practitioners in the medical establishment have advocated the use of the trypsin itself as means of herpes therapy (Szeghy G, Kenyeres B. On the therapy of herpes simplex keratitis with heparin and trypsin. [Article in German] Klin Monatsbl Augenheilkd 1968;153(6):827-30; Sichko ZhV, Kozlova OL. Experience in treating a herpetic infection with trypsin [Article in Russian]. Vrach Delo 1991 March; (3):86-9). This teaching is diametrically opposite to the instant invention.

U.S. Pat. No. 5,532,215 to Lezdey et al., discloses a human-type serine protease inhibitor selected from the group consisting of alpha 1-antitrypsin, secretory leucocyte protease inhibitor (SLPI) and alpha 1-antichymotrypsin. Alpha 1-antitrypsin has been found to be effective in the prevention of the proliferation of viruses, including HSV, which either contain a “chymotrypsin-like amino acid sequence in the nucleocapsid cores” or uses a chymotrypsin-like enzyme as a host environment. Alternatively, Lezdey proposes that AAT can kill viruses on contact. As another alternative it is suggested that other enzymes such as chymotrypsin and aspartyl proteases are involved. No clear teaching has been offered, and the confusing and contradictory statements only serve to teach away from the present invention.

2.2 Serine Protease Inhibitors (Serpins) and Viral Infections

The role of serpins in viral infections other than HIV has been sufficiently documented. For example, PCT publication WO 98/46597 assigned to Emory University (Atlanta, Ga.) discloses complex amino acid containing serpin molecules for treating hepatitis C and herpes viruses including herpes simplex (HSV) and cytomegalovirus (CMV).

The ability of synthetic inhibitors of trypsin-like (TLCK) and chymotrypsin-like (TPCK) proteinases and natural antiproteinase oligopeptides of animal (aprotinin) and microbial (enzistatin) origin to suppress multicycle replication of different alpha viruses (Semliki, Sindbis, Venezuelan equine encephalomyelitis viruses) in cultured cells was found by Zhirnov et al. (Zhirnov O P, Ovcharenko A V, Mel'nikova EE, Gaidamovich SIa, Bukrinskaia A G. Antiviral activity of proteinase inhibitors in cultured cells infected with alpha-viruses. Mol Gen Mikrobiol Virusol 1985 December; (12):30-6). Other similar studies disclose protease inhibitors as useful for a variety of viruses including rotaviruses, myxoviruses (influenza viruses and paramyxoviruses), and herpes virus. Clinically there was significant association between AAT levels and viral infections (Bukrinskaia A G, Kitsak VIa, Moisiadi S A, Arakelov S A. Suppression of rotavirus SA-11 reproduction by protease inhibitors in cell culture. Vopr Virusol 1987 January-February ;32(1):71-4; Chesnokova N B, Maichuk Y F. Antiproteases in herpetic keratitis. Metab Pediatr Syst Ophthalmol 1986;9 (1):593-6; Adelman S F, Howett M K, Rapp F. Protease inhibitors suppress fibrinolytic activity of herpesvirus-transformed cells. J Gen Virol 1982 May; 60(Pt 1): 15-24; Chesnokova N B, Kasavina B S, Maichuk IuF, Kazachenko M A, Shchipanova Al. Main proteolytic inhibitors in ocular herpes. Vopr Med Khim 1981 September-October; 27(5):663-5).

Although the clinical application of protease inhibitors to combat viral infections is quite common, especially in Europe, most of such preparations are aprotinin preparations: Gordox (Gedeon Richter), Contrycal (Germed), Trasylol (Bayer A G), Antagosan (Behring). While aprotinin is a potent inhibitor of kallikrein alpha-2-antiplasmin and alpha-1-antitrypsin are poor inhibitors of kallikrein. A mutant form of AAT inhibitor (alpha-1-l -proteinase inhibitor-Pittsburgh) has been shown to be a more potent inhibitor of kallikrein.

While it was known that patients with cytomegalovirus and bacterial enteritis had raised fecal alpha 1 antitrypsin values this did not lead to suggest or teach that administering AAT might treat or prevent CMV onset (Sharpstone D, Rowbottom A, Nelson M, Gazzard B. Faecal alpha 1 antitrypsin as a marker of gastrointestinal disease in HIV antibody positive individuals. Gut 1996 February; 38(2):206-10).

3. Summary of the Invention

The present invention relates to therapeutically active compounds, pharmaceutical formulations containing said compounds and the use of said compounds in treatment and prophylaxis, particularly of viral infections, more particularly of infections caused by viruses in which said infections are regulated by a serine protease enzyme, especially viruses of the herpes family.

The present invention provides a novel insight into therapy and pathogenesis of viral infection. The present invention equally provides means of prevention of such viral infections. In particular it provides a method of treating viral infection facilitated by a serine proteolytic (SP) activity comprising administering to a subject suffering or about to suffer from said viral infection a therapeutically effective amount of a compound having a serine protease inhibitory or serpin activity comprising α₁-antitrypsin activity (AAT). The viral infection can include retroviral infection such as human immunodeficiency virus (HIV) infection and can also include other unrelated viruses from herpes family such as herpes simplex viruses of type 1 and 2 (HSV), Epstein-Barr virus, Varicella Zoster virus, human herpes viruses type 5, 6, and 8, and cytomegalovirus (CMV) infection. A method of preventing or inhibiting entry of viral nucleic acid into the nucleus of a mammalian host as well as a method of preventing or inhibiting the exit of a virion particle from a mammalian host harboring an agent of a viral infection is provided. Preferably these processes are mediated by endogenous host serine protease (SP) or SP-like activity and will be counteracted by administering a pharmacologically effective amount of a substance exhibiting mammalian alpha-1-antitrypsin (AAT) or AAT-like activity.

Among preferred compounds to treat such viruses is a substantially purified natural or recombinant AAT. Preferably, AAT is substantially purified from a wild type, mutant, or transgenic mammalian source or isolated from a culture of wild type, mutant, or transformed cells.

AAT and similarly active compounds can be identified by a series of assays wherein a compound (AAT) will exhibit inhibitory activity versus control in an assay. One of these assays comprises blocking herpes virus infection in an in vitro model of infection as described in detail in the body of the disclosure.

Also contemplated is a series of peptides comprising carboxyterminal amino acid peptides corresponding to those of AAT. Examples of such peptides include but are not limited to: FVFLM (SEQ. ID NO. 1), FVFAM (SEQ. ID NO. 2), FVALM (SEQ. ID NO. 3), FVFLA (SEQ. ID NO. 4), FLVFI (SEQ. ID NO. 5), FLMII (SEQ. ID NO. 6), FLFVL (SEQ. ID NO. 7), FLFVV (SEQ. ID NO. 8), FLFLI (SEQ. ID NO. 9), FLFFI (SEQ. ID NO. 10), FLMFI (SEQ. ID NO. 11), FMLLI (SEQ. ID NO. 12), FIIMI (SEQ. ID NO. 13), FLFCI (SEQ. ID NO. 14), FLFAV (SEQ. ID NO. 15), FVYLI (SEQ. ID NO. 16), FAFLM (SEQ. ID NO. 17), AVFLM (SEQ. ID NO. 18), FCICV (SEQ. ID NO. 19), FCVCF (SEQ. ID NO. 20), FIVCV (SEQ. ID NO. 21), FCVGV (SEQ. ID NO. 22), FCVLV (SEQ. ID NO. 23), FLVGV (SEQ. ID NO. 24), FSVSV (SEQ. ID NO. 25), FSVCV (SEQ. ID NO. 26), FVCVG (SEQ. ID NO. 27), and combinations thereof.

These peptides can be represented by a general formula (I): N_{T}—X₁—X₂—X₃—X₄l3 X₅—CT or a physiologically acceptable salt thereof, in which N_{T} comprises an amino acid residue positioned at the peptide's N-terminal end, including C, an acetyl group, or a succinyl group, provided that N_{T} can also be absent; X₁ comprises an amino acid residue, including F or A; X₂ comprises an amino acid residue, including C, V, L, M, I, A, C, or S; X₃ comprises an amino acid residue, including F, A, V, M, L, I, Y, or C; X₄ comprises an amino acid residue, including L, A, F, I, V, M, C, G, or S; X₅ comprises an amino acid residue, including M, A, I, L, V, F, or G; and CT comprises an amino acid residue positioned at the peptide's C-terminal end, including C, an amide group, a substituted amide group, or an ester group, provided that CT can also be absent, and in which the amino acid residue can be either an L- or a D-stereoisomeric configuration. These peptides comprise at least 5 amino acids and physiologically acceptable salts thereof. Amino acids in the formula are abbreviated as 1-letter and corresponding 3-letter codes are as follow: Alanine is A or Ala; Arginine R or Arg, Asparagine N or Asn; Aspartic acid Dor Asp; Cysteine C or Cys; Glutamine Q or Gln; Glutamic acid E or Glu; Glycine G or Gly; Histidine H or His; Isoleucine I or Ile; Leucine L or Leu; Lysine K or Lys; Methionine M or Met; Phenylalanine F or Phe; Proline P or Pro; Serine S or Ser; Threonine T or Thr; Tryptophan W or Trp; Tyrosine Y or Tyr; and Valine V or Val.

The peptides of interest are homologous and analogous peptides. While homologues are natural peptides with sequence homology, analogues will be peptidyl derivatives, e.g., aldehyde or ketone derivatives of such peptides. Without limiting to AAT and peptide derivatives of AAT, the compounds like oxadiazole, thiadiazole and triazole peptoids and substances comprising certain phenylenedialkanoate esters are preferred.

The preferred doses for administration can be anywhere in a range between about 10 ng and about 10 mg per ml or mg of the formulation. The therapeutically effective amount of AAT peptides or drugs that have similar activities as AAT or peptides drug can be also measured in molar concentrations and can range between about 1 nM and about 10 mM. The formulation is also contemplated in combination with a pharmaceutically or cosmetically acceptable carrier. The precise doses can be established by well known routine clinical trials without undue experimentation.

Other viral infections are contemplated to be treated, wherein such viral infections are caused by a deficiency in AAT levels or by a dysfunction of AAT. Clinical conditions and viral infections resulting from uncontrolled serine protease activity are also within the scope of the present invention and will be treated alike.

Also a method is contemplated that reduces the likelihood of herpes infection in occupational and non-occupational settings by providing post-exposure prophylaxis. A similar aim of reducing viral infection is accomplished by providing effective antiviral dose of a compound with AAT activity into oral, rectal and/or vaginal cavity to prevent sexual transmission of herpes.

As a derivation of this preferred embodiment a method of reducing or preventing herpes virus replication in a patient is provided which consists of administering a therapeutically effective amount of the instant compound in combination with compounds, e.g., nucleoside drugs like acyclovir, that display anti-herpes activity.

The invention also encompasses methods for the treatment of pre-existing lesions and sores of the skin or mucosa associated with a herpes virus and for prevention of future lesions and sores of the skin or mucosa associated with a herpes virus, which comprise administering the above-described compositions in effective amounts for the treatment and/or prevention of these lesions.

A general method of treating a mammal suffering from a pathological condition that is mediated by viral infection is contemplated as well, which comprises administering a therapeutically effective amount of a substance exhibiting mammalian alpha-1-antitrypsin (AAT) or AAT-like activity. This pathological condition, e.g., inflammatory reaction, tumorigenesis, autoimmune disease, etc., can result directly or indirectly from said viral infections.

Also a method is provided of inhibiting bacterial colonization that occurs concurrently with said viral infection, which comprises administering to a mammalian rendered susceptible to bacterial colonization an effective amount of a substance exhibiting mammalian alpha-l-antitrypsin (AAT) or AAT-like activity. Without limiting to AAT, the compound can be one that inhibits proteinase-3, cathepsin G, or elastase.

Also contemplated is a method of preventing a deficiency of functional endogenous AAT levels in a patient susceptible to a viral infection that is mediated by endogenous host serine protease (SP) or SP-like activity by treating with a pharmaceutical composition in pharmaceutically acceptable carrier comprising effective amounts of a substance exhibiting mammalian alpha-1-antitrypsin (AAT) or AAT-like activity and a thrombolytic agent such as tissue plasminogen activator, urokinase, streptokinase, or combinations or complexes thereof. The pharmaceutical composition can be a peptide or a small molecule, which exhibits AAT or AAT-like activity.

A novel medical treatment and medicine is provided to quickly and safely resolve herpes and other microbial infections. The topical formulation can be applied and maintained on the infected region until the physical symptoms of the disease disappears and the patient is comfortable and has a normal appearance.

Symptoms and diseases to be treated and/or prevented by the instant method include but are not limited to: malaise, fever, chills, rhinitis, diarrhea, atopic eczema, encephalitis, keratoconjunctivitis, pharyngitis, gingivostomatitis, herpetic hepatitis, recurrent orofacial mucocutaneous lesions or herpes labialis, chicken pox skin sores, erythema multiforme, idiopathic burning mouth, aphthous ulceration, Behcet's syndrome, mononucleosis, Burkitt's lymphoma, primary effusion lymphomas, multiple myeloma, angioimmunoblastic lymphadenopathy, Castleman's disease, acquired immune deficiency syndrome (AIDS)-related lymphoma, post-transplantation lymphoproliferative disease, Hodgkin's disease, T-cell lymphomas, oral hairy leukoplakia, lymphoproliferative disease, lymphoepithelial carcinoma, body-cavity-based lymphoma or B-cell lymphomas, non-keratinising carcinoma, squamous cell nasopharyngeal carcinoma, kidney transplant-associated epithelial tumors, malignant mesothelioma, angiosarcoma, Kaposi's sarcoma, angiolymphoid hyperplasia, prostatic neoplasm, cervical cancer, neoplasms of the vulva, retinoblastoma, Li-Fraumeni syndrome, Gardner's syndrome, Werner's syndrome, nervoid basal cell carcinoma syndrome, neurofibromatosis type 1, polyneuropathies, motor neuropathies, sensory neuronopathies, polyradiculoneuropathies, autonomic neuropathies, focal or multifocal cranial neuropathies, radiculopathies, plexopathies typically resulting from tumor infiltration, sexually or perinatally transmitted herpes disease, or combinations thereof.

It should be apparent that in addition to these preferred embodiments a method is contemplated which consists of treating an individual having superficial viral infection or a physiological condition caused, in whole or part, by superficial virus infection of skin, mucosal surface which lines the body cavities. Examples of mucosal surface which can be infected with herpes include infections of the oral soft tissues; middle ear; gastrointestinal tract; urogenital tract; airway/lung tissue, eye; and peritoneal membranes.

In accordance with this embodiment a method of inhibiting topical viral infections or treating topical conditions is provided wherein the target of the therapy are tissues and organs indicated supra and they are contacted with an effective amount of a compound having AAT activity for a sufficient amount of time.

The present inventor discovered that compounds showing AAT activity are potent inhibitors of herpes virus infections. It is therefore the goal of the present invention, in its broadest aspect, to provide methods of treating diseases dependent on the action of protease inhibitors. Accordingly, it should be recognized that this invention is applicable to the control of catalytic activity of serine proteases in any appropriate situation including, but not necessarily limited to, medicine, biology, agriculture, and microbial fermentation. These and other objects and advantages of the present invention will be recognized by those skilled in the art from the following description and representative examples.

Accordingly, it is therefore the overall object of the present invention to provide compounds which exhibit inhibitory activity toward serine proteases.

It is an object of the present invention to provide clinically acceptable serine protease inhibitors with recognized utility and exhibiting relatively high activity at relatively low concentrations.

It is another object of the present invention to provide serine protease inhibitors exhibiting selectivity for clearly defined key proteases involved in viral activation and infection.

The present invention encompasses topical pharmaceutical compositions for the treatment of pre-existing lesions and sores of the skin or mucosa associated with a herpes virus and for prevention of future lesions and sores of the skin or mucosa associated with a herpes virus. The compositions comprise agents exhibiting AAT activity.

These and other objects and advantages of the present invention will be recognized by those skilled in the art from the following description and illustrative examples.

### 4. BRIEF DESCRIPTION OF THE DRAWINGS

FIG. 1 illustrates effect of AAT on CMV production.

FIG.2 illustrates effect of AAT on HSV production.

### 5. Glossary

As used hereinafter the term “herpes virus” is a generic term which applies equally to any known or to be known viruses of the herpes family including but not limited to HSV-1, HSV-2, CMV, EBV, VZV, HHV-5, HHV-6, HHV-8, and combination thereof.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, a “pharmaceutical carrier” is a pharmaceutically acceptable solvent, suspending agent, liposome or vehicle for delivering the anti-cancer agent to the animal or human. The carrier can be liquid or solid and is selected with the planned manner of administration in mind.

As used herein, “viruses” includes viruses which cause diseases in warm blooded animals including HIV, influenza, rhinoviruses, herpes and the like.

As used herein, “suspensions” are dispersions of solid particles in a liquid continuous phase with or without the aid of a suspending agent. As used herein, “emulsions” are a dispersion of two immiscible liquids. One liquid is dispersed as small globules in the other liquid with the aid of an emulsifying agent. As used herein, “lotions” are liquids that are intended for topical or external application to the skin and can contain suspended solid particles. As used herein, “ointments” and/or “creams” are semi-solid preparations intended for application to the skin. They can consist of oleaginous substances or can be free from oleaginous substances.

### 6. DETAILED DESCRIPTION OF THE INVENTION

This invention is directed to a method for treating and/or preventing conditions caused by viruses from the herpesviridae family. While the invention will now be described in connection with certain preferred embodiments in the following examples so that aspects thereof can be more fully understood and appreciated, it is not intended to limit the invention to these particular embodiments. On the contrary, it is intended to cover all alternatives, modifications and equivalents as can be included within the scope of the invention as defined by the appended claims. Thus, the following examples which include preferred embodiments will serve to illustrate the practice of this invention, it being understood that the particulars shown are by way of example and for purposes of illustrative discussion of preferred embodiments of the present invention only and are presented in the cause of providing what is believed to be the most useful and readily understood description of formulation procedures as well as of the principles and conceptual aspects of the invention.

This invention relates to the identification, purification and manipulation of viral proteases for the development of methodology and compositions for the treatment and prevention of viral infections. The proteases of the present invention can be further defined as serine proteases with the properties expected of this category of protease. A serine protease is an enzyme, which catalyzes the hydrolysis of peptide bonds, and typically have a serine residue at the active site. Serine proteases also typically include an arrangement of a triad of catalytic residues that are somewhat removed from one another in the linear arrangement of amino acids, but brought together as a “proteolytic cleft” in the properly folded protease. Various differences have been observed in this catalytic triad from protease to protease. For example, in both trypsin and subtilisin serine proteases, Asp, His, and Ser are the amino acids of the catalytic triad. However, in trypsin-like serine proteases, they are arranged His, Asp, Ser, whereas subtilisin-like proteases are arranged Asp, His, Ser. There are also differences in the relative spacing of these key residues. In addition, there are other evolutionarily conserved features of these proteases, which allow them to be identified as serine proteases and subsequently classified. The presence of the catalytically important Asp, His, and Ser residues are the crucial tests, regardless of the membership and classification in the serine proteases adopted by various sources and authorities.

The proteases appear to be essential for development of the capsid of the virus. Consequently, inhibiting the protease action will lead to disruption of the lytic cycle of the virus. Such proteases are thus optimal targets for antiviral therapy. In particular, the target is useful for attacks on the herpes virus.

Furthermore this invention addresses protease of the host cell and enzymes of similar nature present in the fluid of intercellular space, e.g., serum, saliva, plasma, blood, urine, pus, tears, urine, semen, vaginal secretion, spinal fluid, etc.

AAT can also be administered in effective dosage to suppress inflammation processes associated or caused by viral infections, for example, inflammation processes that occur in the eye as a consequence of HSV-1 infection. Inflammation is a typical pathological process (i.e., either inherent in or associated with a variety of distinct diseases and illnesses), defensive in nature, but potentially dangerous if uncontrolled. There are several indices of inflammation at the organism level: e.g., disorders such as hyperemia, edema, pain, fever, and sores. At the cellular level, inflammation is characterized by leukocyte migration into affected tissues. At the molecular level, inflammation is characterized by activation of various defense molecules, e.g., complement, histamine, kinin, lymphokines, cytokines, and eicosanoids. When inflammation is generalized, the various indices of inflammation can become disseminated and occur throughout the entire organism. Without wishing to be limited to any particular mechanism of operation of AAT, the beneficial effect of AAT is provided due to it's capacity to act as an agent regulating inflammation or acute reaction. Particularly preferred are small (low molecular weight) agents which are either peptides or non-peptide designer molecules imitating AAT activity.

The invention also provides a topical pharmaceutical composition for the prevention and treatment of lesions and sores of the skin, mucous membranes, or mucosa associated with a herpes viruses, comprising AAT and mimics thereof as active ingredients therein, in combination with a pharmaceutically or cosmetically acceptable carrier.

The invention also provides a method for treating a superficial herpes virus infection, and for reducing the likelihood of future superficial herpes virus infection, comprising administering a topical pharmaceutical composition having an effective amount of AAT or a compound with AAT activity and a pharmaceutically acceptable or a cosmetically acceptable carrier.

Preferably, said topical composition will comprise a polyhydroxy compound selected from the group consisting of glycerine, propylene glycol, and polyethylene glycol. More specifically, the present invention provides a topical pharmaceutical composition, wherein said antiviral agent comprises agents with AAT like activity. Thus, the present invention, in especially preferred embodiments, provides a composition containing between about 0.1% and about 30% of said active ingredient. The invention also provides a method for the prevention and treatment of lesions and sores of the skin or mucosa associated with a herpes virus, comprising administering a topical pharmaceutical composition, having AAT mimicking agent as an active ingredient therein, in an effective amount for the treatment of lesions and sores of the skin or mucosa, in combination with a pharmaceutically or cosmetically acceptable carrier. Such a composition can be effective for a wide range of virus-associated and viral-like diseases. These include herpes simplex labialis, post-herpetic neuralgia, recurrent genital herpes, blepharitis, cancer sore, aphthous stomatitis, vulvar vestibulities, etc.

Further still, another preferred embodiment is the treatment of the following herpes-caused eye conditions or diseases, which can be either acute or chronic: allergic conjunctivitis, keratoconjuctivitis, conjunctivitis, blepharitis, retinitis, edema, epithelial keratitis, iridocyclitis, scleritis, trachoma, uveitis, opacification, ulceration, and inflammation associated therewith as secondary sequelae of primary autoimmune or non-autoimmune diseases. Representative primary autoimmune diseases include ulcerative colitis, Mooren's ulcer, psoriasis, systemic lupus erythematosus, rheumatoid arthritis, Wegener's granulomatosis, polyarteritis nodosa, or myasthenia gravis. Representative non-autoimmune diseases include ocular dystrophies such as macular dystrophy and Fuch's dystrophy.

While, as indicated, it has been discovered that the above composition is effective in itself, the carboxylic acid salt of the present invention can obviously also be combined in a pharmaceutical composition with an additional poorly soluble antiviral nucleoside derivative, such as acyclovir, vidarabine, azidothymidine and ganciclovir.

The pharmaceutically or cosmetically acceptable vehicle utilizable in the compositions of the present invention can be selected from the group comprising an oil-in-water or water-in-oil emulsion, solution, cream, suspension, gel, aerosol, or powder.

Oil-in-water or water-in-oil emulsions are formulated such that a stable topical ointment, lotion, cream, stick or foam is achieved. The stabilization of the topical emulsions can be established and optimized by using the preferred combinations of hydrophilic and lipophilic emulsifiers, properly aligned at the water/oil interface. The emulsifying agents and their concentrations and proportions can be chosen according to the principle of the well-established HLB method published by W. C. Griffen, “H. L. B.—The Hydrophilic-Lipophilic Balance,” J. Soc. Cos. Met. Chem., Vol. 5, p. 249 (1954).

In the case where the composition according to the invention is an emulsion, an oil phase is selected. Examples of suitable oil phase include but are not limited to beeswax, spermaceti, 2-octyl dodecanol, lanolin, sodium C.sub. 12-15 alcohols sulphate, esters of fatty acids and high molecular weight alcohols such as cetyl palmitate and cetearyl octanoate, esters of fatty acids and branched alcohols or polyols such as isopropyl palmitate or myristate, cocoglycerides, cosbiol, wool alcohols, cocoa butter, stearyl alcohol, cholesterol, liquid paraffin, soft paraffin, hard paraffin, or the like.

Examples of the emulsifying agents used for the purpose of dispersion of the above-mentioned fats or oils and the like in the aqueous phase include non-ionic surfactants, such as sorbitan sesquioleate, PEG-5 glyceryl stearate, poloxamers, cetostearyl alcohol, polysorbate 60, sorbitan monostearate, sorbitan monooleate, and glyceryl monostearate.

In the case where the composition according to the invention is a gel or solution, the composition preferably contains an oleic acid/oleate salt, and generally a lower alkanol having from one to four carbon atoms, water, a gelifying agent (if a gel), one or more polyhydric alcohols selected from the group consisting of a lower alkylene glycol having from two to four carbon atoms, glycerine, and polyethylene glycol, having an average molecular weight from 200 to 2000, and a base, e.g., sodium hydroxide, or an acid, e.g., citric acid, for pH adjustment.

Examples of gelifying agents include polysaccharides such as cellulose derivatives, acrylic polymers, proteins, polyhydroxy compounds such as polyethylene glycol having an average molecular weight from 400 to 2000, and polyoxyethylene-3-cetylstearyl alcohol, known as Emulgin B3.

All semi-solid topical preparations should preferably be stable and consistent, non-leaky, non-staining, and non-greasy.

In the case where the composition according to the present invention is a powder, the composition preferably contains an oleic acid and/or alkali oleate, and generally a diluting powder compound suitable as a lubricant. This lubricant is selected from the group consisting of talc, microcrystalline cellulose, polyvinyl pyrrolidone, metal stearates, lactose or starch known to have non-irritating, non-toxic and inert properties.

In accordance with another aspect of the invention, the oleic acid and/or oleate salt could be topically applied in a slow-release manner using an adhesive sponge bandage, or, alternatively, a gauze or sponge sandwich containing a layer of the active principals of the invention situated between upper and lower absorbent layers.

The carboxylic/dicarboxylic acids and/or their salts of the present invention can also be applied onto the mucosa, for example, as a buccal gel or vaginal preparation. For this purpose, several bioadhesive polymers are chosen. Examples of such bioadhesive polymers include polyethylene glycols, cellulose derivatives, starch, and polyacrylic acid such as polycarbophil and Carbopol 934.

As described hereinbefore, the vehicles can be in the form of a cream, lotion, ointment, gel, stick, topical solution, gargle solution, foam, spray, liquid soap, or powder. From the point of view regarding the formulation characteristics, the pharmaceutical preparations could be processed as a water-in-oil or an oil-in-water emulsion, clear solution, gel solution, aerosol, powder mix, film-forming liquid, bioadhesive preparation, detergents-containing gel, suspension in gel, liquid, or emulsion, etc.

The peptide-based serine protease inhibitors can be prepared by any suitable synthesis method such as originally described by Merrifield, J. Am. Chem. Soc., lb 85, p 2149 (1963). Synthetic peptides which exhibit inhibitory activity toward serine proteases and methods for preparing and using same are disclosed for example in U.S. Pat. Nos. 4,829,052, 5,157,019 to Glover; U.S. Pat. No. 5,420,110 to Miller; U.S. Pat. No. 4,963,654 Katunuma each incorporated herein by reference in its respective entirety.

Those skilled in the art of biochemical synthesis will recognize that for commercial-scale quantities of peptides, such peptides are preferably obtained using recombinant DNA techniques.

It is to be understood that the present invention is not limited to the examples described hereinabove and other serine protease inhibitors known in the art can be used within the limitations of the invention. For example, one skilled in the art can easily adopt inhibitors as described in WO 98/24806, which discloses substituted oxadiazole, thiadiazole and triazole as serine protease inhibitors. U.S. Pat. No. 5,874,585 discloses substituted heterocyclic compounds useful as inhibitors of serine proteases; U.S. Pat. No. 5,869,455 N-substituted derivatives; U.S. Pat. No. 5,861,380 protease inhibitors-keto and di-keto containing ring systems; U.S. Pat. No. 5,807,829 serine protease inhibitor-tripeptoid analogues; U.S. Pat. No. 5,801,148 serine protease inhibitors-proline analogues; U.S. Pat. No. 5,618,792 substituted heterocyclic compounds useful as inhibitors of serine proteases. These patents and PCT publications and others as listed infra are incorporated herein by reference. Other equally advantageous molecules, which can be used instead of AAT or in combination with AAT, are contemplated such as in WO 98/20034 disclosing serine protease inhibitors from fleas. Without limiting to this single reference one skilled in the art can easily and without undue experimentation adopt compounds such as in WO98/23565 which discloses aminoguanidine and alkoxyguanidine compounds useful for inhibiting serine proteases; WO98/50342 discloses bis-aminomethylcarbonyl compounds useful for treating cysteine and serine protease disorders; WO98/50420 cyclic and other amino acid derivatives useful for thrombin-related diseases; WO 97/21690 D-amino acid containing derivatives; WO 97/10231 ketomethylene group-containing inhibitors of serine and cysteine proteases; WO 97/03679 phosphorous containing inhibitors of serine and cysteine proteases; WO 98/21186 benzothiazo and related heterocyclic inhibitors of serine and proteases; WO 98/22619 discloses a combination of inhibitors binding to P site of serine proteases with chelating site of divalent cations; WO 98/22098 a composition which inhibits conversion of pro-enzyme CPP32 subfamily including caspase 3 (CPP32/Yama/Apopain); WO 97/48706 pyrrolo-pyrazine-diones; WO 97/33996 human placental bikunin (recombinant) as serine protease inhibitor; WO 98/46597 complex amino acid containing molecule for treating viral infections and conditions disclosed hereinabove.

The pentapeptides of the present invention can form a salt by acid addition. For example, the polypeptide forms a salt with an inorganic acid (hydrochloric acid, hydrobromic acid, phosphoric acid, nitric acid, sulfuric acid or the like) or an organic carboxylic acid (acetic acid, halo acetic acid such as trifluoroacetic acid, propionic acid, maleic acid, succinic acid, malic acid, citric acid, tartaric acid, salicylic acid and an acidic sugar (glucuronic acid, galacturonic acid, gluconic acid, ascorbic acid or the like), an acidic polysaccharide (hyaluronic acid, chondroitin sulfate, alginic acid or the like) or an organic sulfonic acid (methanesulfonic acid, p-toluenesulfonic acid or the like) including sulfonic acid sugar ester such as chondroitin sulfates.

To provide even better protection and curative effect, compounds of the invention can be administered in combination with other antiviral agents, such as acyclovir, valcyclovir, penciclovir, famciclovir, ganciclovir and its prodrugs, cidofovir, foscarnet and the like for indications that are caused by herpes viruses in general.

Without limiting to nucleoside anti-herpes drugs the present invention provides a combination therapy with other antivirally effective compounds known in the art. For example active ingredients in plants known to have anti-herpes activity. The phytotherapeutic agent can consist of an extract of components extractable from sage, (Salvia officinalis), ribwort plantain (Plantago lanceolata), greater plantain (Plantago major), mistletoe (Viscum album), absinthe (Artemisia absinthium), resin of mastic (resin Pistacia lenticus), fruit of Delphinium denudatum, rose buds (Flores rosae), seeds of cardamom (Fructus ellettaria cardamomum, borage flowers (Flores onosma brateatum boriginaceae), Phytolacca decandra, Hypericum perforatum, Isatides tinctoria radix, Isatides tinctoria folium, Pueraria lobata radix, Forsythia suspensa fructus, Lonicera japonica flos, and Chrysanthemem indici flos, in a pharmacologically effective amount. This combination method can further comprise administering the instant therapeutic agent with non-antiviral drugs such as anesthetics, including for example, benzocaine, procaine, propoxycaine, dibucaine and lidocaine, as well as non-antiviral drugs such as analgesics, antipyretics, sedatives, antibiotics, and combinations thereof

The compounds of the invention can be administered at a daily dose generally in the range 0.1 to 200 mg/kg/day, advantageously, 0.5 to 100 mg/kg/day, more preferably 10 to 50 mg/kg/day, such as 10 to 25 mg/kg/day. A typical dosage rate for a normal adult will be around 50 to 500 mg, for example 300 mg, once or twice or as many as 10 times per day.

The compounds of the invention can be administered orally, but can also be administered rectally, vaginally, nasally, by inhalation, topically, transdermally or parenterally, for instance intramuscularly, intravenously or epidurally. The compounds can be administered alone, for instance in a capsule, but will generally be administered in conjunction with a pharmaceutically acceptable carrier or diluent. The invention extends to methods for preparing a pharmaceutical composition comprising bringing the instant compound or its pharmaceutically acceptable salt/mimics in conjunction or association with a pharmaceutically acceptable carrier or vehicle.

Oral formulations are conveniently prepared in unit dosage form, such as capsules or tablets, employing conventional carriers or binders such as magnesium stearate, chalk, starch, lactose, wax, gum or gelatin. Liposomes or synthetic or natural polymers such as HPMC or PVP can be used to afford a sustained release formulation. Alternatively the formulation can be presented as a nasal or eye drop, syrup, gel or cream comprising a solution, suspension, emulsion, oil-in-water or water-in-oil preparation in conventional vehicles such as water, saline, ethanol, vegetable oil or glycerine, optionally with flavourant and/or preservative and/or emulsifier.

The compounds of the present invention are used as therapeutic agents in the treatment of a physiological (especially pathological) conditions caused in whole or part, by uncontrolled serine protease activity. The peptides can be administered as free peptides or pharmaceutically acceptable salts thereof. The terms used herein conform to those found in Budavari, Susan (Editor), “The Merck Index” An Encyclopedia of Chemicals, Drugs, and Biologicals; Merck & Co., Inc. The term “pharmaceutically acceptable salt” refers to those acid addition salts or metal complexes of the peptides which do not significantly or adversely affect the therapeutic properties (e.g. efficacy, toxicity, etc.) of the peptides. The peptides should be administered to individuals as a pharmaceutical composition, which, in most cases, will comprise the peptide and/or pharmaceutical salts thereof with a pharmaceutically acceptable carrier. The term “pharmaceutically acceptable carrier” refers to those solid and liquid carriers, which do not significantly or adversely affect the therapeutic properties of the peptides. The pharmaceutical compositions containing peptides of the present invention can be administered to individuals, particularly humans, either intravenously, subcutaneously, intramuscularly, intranasally or even orally. The necessary dosage will vary with the particular condition being treated, method of administration and rate of clearance of the peptide from the body. In most cases dosages between 0.001 and 30 mg/kg should be effective. A dose range between 0.1 and 10 mg/ml of bodily fluid, such as blood, plasma, serum, semen, or saliva is preferred.

Routes of administration include, but are not limited to, topical, transdermal, parenteral, gastrointestinal, transbronchial and transalveolar. Topical administration is accomplished via a topically applied cream, gel, rinse, etc. containing therapeutically effective amounts of serpins. Transdermal administration is accomplished by application of a cream, rinse, gel, etc. capable of allowing the serpins to penetrate the skin and enter the blood stream. Parenteral routes of administration include, but are not limited to, direct injection such as intravenous, intramuscular, intraperitoneal or subcutaneous injection. Gastrointestinal routes of administration include, but are not limited to, ingestion and rectal. Transbronchial and transalveolar routes of administration include, but are not limited to, inhalation, either via the mouth or intranasally and direct injection into an airway, such as through a tracheotomy.

Although the compounds described herein and/or their derivatives can be administered as the pure chemicals, it is preferable to present the active ingredient as a pharmaceutical composition. The invention thus further provides the use of a pharmaceutical composition comprising one or more compounds and/or a pharmaceutically acceptable salt thereof, together with one or more pharmaceutically acceptable carriers therefor and, optionally, other therapeutic and/or prophylactic ingredients. The carrier(s) must be acceptable in the sense of being compatible with the other ingredients of the composition and not deleterious to the recipient thereof.

Pharmaceutical compositions include those suitable for oral or parenteral (including intramuscular, subcutaneous and intravenous) administration. The compositions can, where appropriate, be conveniently presented in discrete unit dosage forms and can be prepared by any of the methods well known in the art of pharmacy. Such methods include the step of bringing into association the active compound with liquid carriers, solid matrices, semi-solid carriers, finely divided solid carriers or combination thereof, and then, if necessary, shaping the product into the desired delivery system.

Pharmaceutical compositions suitable for oral administration can be presented as discrete unit dosage forms such as hard or soft gelatin capsules, cachets or tablets, each containing a predetermined amount of the active ingredient; such as a powder or as granules; as a solution, a suspension or as an emulsion. The active ingredient can also be presented as a bolus, electuary or paste. Tablets and capsules for oral administration can contain conventional excipients such as binding agents, fillers, lubricants, disintegrants, or wetting agents. The tablets can be coated according to methods well known in the art, e.g., with enteric coatings.

Oral liquid preparations can be in the form of, for example, aqueous or oily suspension, solutions, emulsions, syrups or elixirs, or can be presented as a dry product for constitution with water or another suitable vehicle before use. Such liquid preparations can contain conventional additives such as suspending agents, emulsifying agents, non-aqueous vehicles (which can include edible oils), or preservative.

The compounds can also be formulated for parenteral administration (e.g., by injection, for example, bolus injection or continuous infusion) and can be presented in unit dose form in ampules, pre-filled syringes, small bolus infusion containers or in multi-does containers with an added preservative. The compositions can take such forms as suspensions, solutions, or emulsions in oily or aqueous vehicles, and can contain formulatory agents such as suspending, stabilizing and/or dispersing agents. Alternatively, the active ingredient can be in powder form, obtained by aseptic isolation of sterile solid or by lyophilization from solution, for constitution with a suitable vehicle, e.g., sterile, pyrogen-free water, before use.

For topical administration to the epidermis, the compounds can be formulated as ointments, creams or lotions, or as the active ingredient of a transdermal patch. Suitable transdermal delivery systems are disclosed, for example, in Fisher et al. (U. S. Pat. No. 4,788,603) or Bawas et al. (U. S. Pat. Nos. 4,931,279, 4,668,504 and 4,713,224). Ointments and creams can, for example, be formulated with an aqueous or oily base with the addition of suitable thickening and/or gelling agents. Lotions can be formulated with an aqueous or oily base and will in general also contain one or more emulsifying agents, stabilizing agents, dispersing agents, suspending agents, thickening agents, or coloring agents. The active ingredient can also be delivered via iontophoresis, e.g., as disclosed in U. S. Pat. Nos. 4,140,122, 4,383,529, or 4,051,842. At least two types of release are possible in these systems. Release by diffusion occurs when the matrix is non-porous. The pharmaceutically effective compound dissolves in and diffuses through the matrix itself. Release by microporous flow occurs when the pharmaceutically effective compound is transported through a liquid phase in the pores of the matrix.

Compositions suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; mucoadherent gels, and mouthwashes comprising the active ingredient in a suitable liquid carrier.

When desired, the above-described compositions can be adapted to provide sustained release of the active ingredient employed, e.g., by combination thereof with certain hydrophilic polymer matrices, e.g., comprising natural gels, synthetic polymer gels or mixtures thereof.

The pharmaceutical compositions according to the invention can also contain other adjuvants such as flavorings, coloring, antimicrobial agents, or preservatives.

It will be further appreciated that the amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, the compound is conveniently administered in unit dosage form; for example, containing 5 to 2000 mg, conveniently 10 to 1000 mg, most conveniently, 50 to 500 mg of active ingredient per unit dosage form.

Ideally, the active ingredient should be administered to achieve peak plasma concentrations of the active compound of from about 100 ng to 10 mg, preferably, about 1 microgram to 5 mg most preferably, about 2 to about 4 mg. This can be achieved, for example, by the intravenous injection of a 0.05 to 5% solution of the active ingredient, optionally in saline, or orally administered as a bolus containing about 1-100 mg of the active ingredient. Desirable blood levels can be maintained by continuous infusion to provide about 0.01-5.0 mg/kg/hr or by intermittent infusions containing about 0.4-20 mg/kg of the active ingredient(s). Buffers, preservatives, antioxidants and the like can be incorporated as required.

The desired dose can conveniently be presented in a single dose or as divided doses administered at appropriate intervals, for example, as two, three, four or more sub-doses per day. The sub-dose itself can be further divided, e.g., into a number of discrete loosely spaced administrations, such as multiple inhalations from an insulator or by application of a plurality of drops into the eye.

### 7. EXAMPLES

Data are presented as means ±SEM. Group means are compared by ANOVA using Fisher's least significant difference. For data expressed as percent change, the values for p24 in control cultures (medium alone) are subtracted from those for each culture-containing stimulus. The p24 concentrations in cultures conducted in the presence of stimulus alone are set at 100%. Percent p24 in cultures containing stimulus and AAT are calculated by dividing the measured p24 by that present in cultures containing stimulus alone. The resultant fraction is expressed as a percent.

### 7.1 Example 1.Activity Against HSV In Vitro

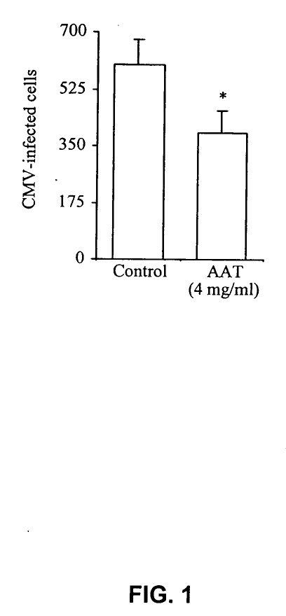

Preparations of HSV-1 and HSV-2 are obtained from a commercial source (ATCC) and from clinical isolates. Semi-continuous human lung fibroblast (HLF) cells are seeded in a 96-well plate and exposed to a 100-dilution of inoculum of each virus strain and allowed to absorb in the presence or absence of AAT. The virus is then removed and fresh medium is added. Cultures are incubated and inspected regularly by microscopy for evidence of virus growth. The culture medium is normally changed on the day after inoculation and is then replaced periodically to replenish the supply of nutrients for the cells. Cultures are incubated for various lengths of time depending on the virus. The cytopathic effects of a concentrated inoculum of herpes virus appears overnight. The effect of AAT on HSV is measured by an ELISA using rabbit anti-HSV antibody (Accurate, Westbury, N.Y.) and results are quantified based on optical density of horse radish peroxidase reaction and expressed as a percent of a control. The inhibitory effect of AAT preparation is shown in FIG. 1. AAT displays significant inhibitory effect (as indicated by asterisks).

### 7.2 Example 2. Activity Against CMV In Vitro

Preparations of CMV are obtained from a commercial source (ATCC) and from clinical isolates. Semi-continuous human lung fibroblast (HLF) cells are seeded in a 96-well plate and exposed to a 100-dilution of inoculum of each CMV isolate and allowed to absorb in the presence or absence of AAT. Virus is then removed and fresh medium is added. Cultures are incubated at 37° C. and are inspected regularly by microscopy for evidence of virus growth. The culture medium is normally changed on the day after inoculation and is then replaced periodically to replenish the supply of nutrients for the cells. Cultures are incubated for various lengths of time depending on the virus. Usually, slow replicating cytomegalovirus takes a week or more to appear. The CMV assay is based on a standard plaque reduction assay on day 6-10 post-infection. Cells are first fixed with 10% formaldehyde and then stained with 0.8% Crystal Violet, and plaques are counted under microscope. The percent of reduction in number of plaques is recorded as a function of AAT concentration present in culture wells.

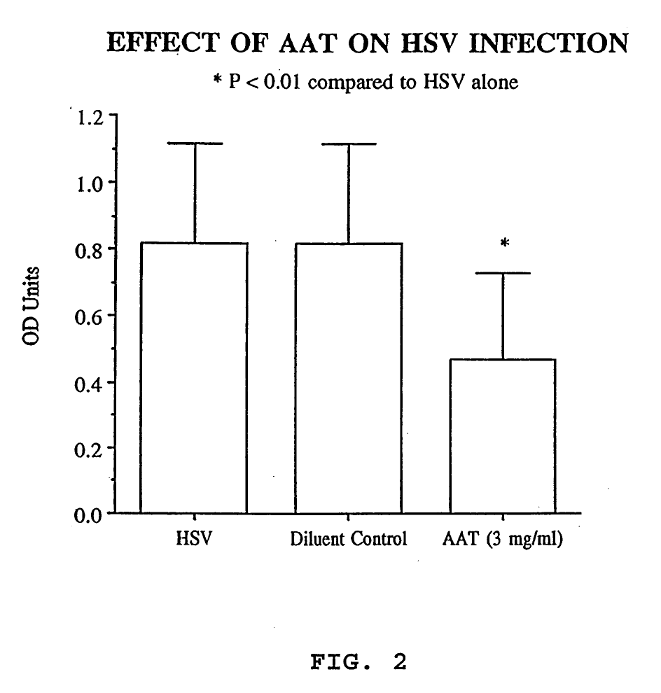

The inhibitory effect of AAT preparation is shown in FIG. 2. AAT shows significant inhibitory effect.

### 7.3 Example 3. Antiviral Activity In Vivo

Herpes simplex virus-2 (HSV-2 )-infected SCID mouse is an animal model to determine the efficacy of disclosed antiviral agents in vivo. Mice are approximately 6 to 9 weeks old and weigh approximately 20 to 28 grams at the time of test initiation. All mice used in the study do not vary in age by more than 10 days. The mice are housed 6 per cage with bedding. The mice are fed rodent diet 5002 (PMI, St. Louis Mo.) ad libitum. Fresh water is supplied to the mice ad libitum. Herpes simplex virus-2, strain MS, is used to challenge the mice. Prior to infectious challenge a vial of frozen stock is thawed and diluted to the appropriate concentration in buffered saline solution. The mice are anesthetized with Halothane and the virus challenge dose is rubbed into abraded skin on the back of mice in volume of 50 microlitres. SCID mice inoculated with HSV-2 at 1000 times the LD₅₀ are administered either with a topical formulation comprising the currently marketed anti-herpes agent acyclovir three times daily in a suitable vehicle or the compound of Example 1 or 2 (100 mg/kg) in the same vehicle three times daily for 7 consecutive days beginning 2 hours after inoculation. The animals are assessed daily for deaths. The percentage of mice surviving the HSV-1 infection is significantly greater following a given dose of the compound of the invention relative to control administration consisting of vehicle without drug. The mice treated with the instant compound having AAT activity and acyclovir both result in about 60% survival rate, while the untreated control group show a 20% survival rate. Similar improved survival rates are obtained with a representative peptide of the invention having sequence FVYLI (SEQ. ID NO. 16). Among those several are equally acceptable including FVFLM (SEQ. ID NO. 1), FVFAM (SEQ. ID NO. 2), FVALM (SEQ. ID NO. 3), FVFLA (SEQ. ID NO. 4), FLVFI (SEQ. ID NO. 5), FLMII (SEQ. ID NO. 6), FLFVL (SEQ. ID NO. 7), FLFVV (SEQ. ID NO. 8), FLFLI (SEQ. ID NO. 9), FLFFI (SEQ. ID NO. 10), FLMFI (SEQ. ID NO. 11), FMLLI (SEQ. ID NO. 12), FIIMI (SEQ. ID NO. 13), FLFCI (SEQ. ID NO. 14), FLFAV (SEQ. ID NO. 15), FAFLM (SEQ. ID NO. 17), AVFLM (SEQ. ID NO. 18), FCICV (SEQ. ID NO. 19), FCVCF (SEQ. ID NO. 20), FIVCV (SEQ. ID NO. 21), FCVGV (SEQ. ID NO. 22), FCVLV (SEQ. ID NO. 23), FLVGV (SEQ. ID NO. 24), FSVSV (SEQ. ID NO. 25), FSVCV (SEQ. ID NO. 26), FVCVG (SEQ. ID NO. 27), and combinations thereof.

These peptides are represented by a general formula (I): N_{T}—X₁ —X₂—X₃—X₄—X₅—C_{T} or a physiologically acceptable salt thereof, in which N_{T} comprises an amino acid residue positioned at the peptide's N-terminal end, including C, an acetyl group, or a succinyl group, provided that N_{T} can also be absent; X₁ comprises an amino acid residue, including F or A; X₂ comprises an amino acid residue, including C, V, L, M, I, A, C, or S; X₃ comprises an amino acid residue, including F, A, V, M, L, I, Y, or C; X₄ comprises an amino acid residue, including L, A, F, I, V, M, C, G, or S; X₅ comprises an amino acid residue, including M, A, I, L, V, F, or G; and C_{T} comprises an amino acid residue positioned at the peptide's C-terminal end, including C, an amide group, a substituted amide group, or an ester group, provided that C_{T} can also be absent, and in which the amino acid residue can be either an L- or a D-stereoisomeric configuration. These peptides comprise at least 5 amino acids and physiologically acceptable salts thereof. The peptides of interest are homologous and analogous peptides. While homologues are natural peptides with sequence homology, analogues will be peptidyl derivatives, e.g., aldehyde or ketone derivatives of such peptides. Anti-herpes effective doses of these peptides are in a range from about 1 mg/kg to approximately 100 mg/kg.

### 7.4 Example 4. Antiviral Activity of Man-Made Small Molecules

Without limiting to AAT and peptide derivatives of AAT, the compounds like oxadiazole, thiadiazole and triazole peptoids are preferred as they also show an equivalent antiviral activity in a mouse model as described in above Example 3. Anti-herpes effective doses are in a range from about 1 μg/kg to approximately 100 mg/kg. Specific examples of such oxadiazole, thiadiazole and triazole peptoids are molecules such as Benzyloxycarbonyl-L-valyl-N-[1-(2-(3-methylbenzyl)-1,3,4-oxadiazolyl]carbonyl)-2-(S)-methylpropyl]-L-prolinamide; Benzyloxycarbonyl-L-valyl-N-[1-2-(5-(methyl)-1,3,4-oxadiazoly]carbony)-2-(S)-methylpropyl]-L-prolinamide; Benzyloxycarbonyl)-L-valyl-N-[1-(2-(5-(3-trifluoromethylbenzyl)-1,3,4-oxadiazolyl]carbonyl)-2-(S)-methylpropyl]-L-prolinamide; Benzyloxycarbonyl)-L-valyl-N-[1-(2-(5-(4-Dimethylaminobenzyl)-1,3,4-oxadiazolyl]carbonyl)-2-(S)-methylpropyl]-L-prolinamide; Benzyloxycarbonyl)-L-valyl-N-[1-(2-(5-(1-napthylenyl)-1,3,4-oxadiazolyl]carbonyl)-2-(S)-methylpropyl]-L-prolinamide; (Benzyloxycarbonyl)-L-valyl-[1-(3-(5-(3,4-methylenedioxybenzyl)-1,2,4-oxadiazolyl]carbonyl)-2-(S)-methylpropyl]-L-prolinamide; Benzyloxycarbonyl)-L-valyl-N-[1-(3-(5-(3,5-dimethylbenzyl)-1,2,4-oxadiazolyl]carbonyl)-2-(S)-methylpropyl]-L-prolinamide; (Benzyloxycarbonyl )-L-valyl-N-[1-(3-(5-(3,5-dimethoxybenzyl)-1,2,4-oxadiazolyl]carbonyl)-2-(S)-methylpropyl]-L-prolinamide; (Benzyloxycarbonyl)-L-valyl-N-[1-(3-(5-(3,5-ditrifluoromethylbenzyl)-1,2,4-oxadiazolyl]carbonyl)-2-(S)-methylpropyl]-L-prolinamide; (Benzyloxycarbonyl)-L-valyl-N-[1-(3-(5-(3-methylbenzyl)-1,2,4-oxadiazolyl]carbonyl)-2-(S)-methylpropyl]-L-prolinamide; (Benzyloxycarbonyl)-L-valyl-N-[1-(3-(5-(biphenylmethine)-1,2,4-oxadiazolyl]carbonyl)-2-(S)-methylpropyl]-L-prolinamide; (Benzyloxycarbonyl)-L-valyl-N-[1-(3-(5-(4-phenylbenzyl )-1,2,4-oxadiazolyl]carbonyl)-2-(S)-methylpropyl]-L-prolinamide; (Benzyloxycarbonyl)-L-valyl-N-[1-(3-(5-(3-phenylbenzyl)-1,2,4-oxadiazolyl]carbonyl)-2-(S)-methylpropyl]-L-prolinamide; (Benzyloxycarbonyl)-L-valyl-N-[1-(3-(5-(3-phenoxybenzyl)-1,2,4-oxadiazolyl]carbonyl)-2-(S)-methylpropyl]-L-prolinamide; (Benzyloxycarbonyl)-L-valyl-N-[1-(3-(5-(cyclohexylmethylene)-1,2,4-oxadiazolyl]carbonyl)-2-(S)-methylpropyl]-L-prolinamide; (Benzyloxycarbonyl)-L-valyl-N-[1-(3-(5-(3-trifluoromethyldimethylmethylene)-1,2,4-oxadiazolyl]carbonyl)-2-(S)-methylpropyl]-L-prolinamide; (Benzyloxycarbonyl)-L-valyl-N-[1-(3-(5-(1-napthylmethylene)-1,2,4-oxadiazolyl]carbonyl)-2-(S)-methylpropyl]-L-prolinamide; (Benzyloxycarbonyl)-L-valyl-N-[1-(3-(5-(3-pyridylmethyl)-1,2,3--oxadiazolyl]carbonyl)-2-(S)-methylpropyl]-L-prolinamide; (Benzyloxycarbonyl)-L-valyl-N-[1-(3-(5-(3,5-diphenylbenzyl)-1,2,4-oxadiazolyl]carbonyl)-2-(S)-methylpropyl]-L-prolinamide; (Benzyloxycarbonyl)-L-valyl-N-[1-(3-(5-(4-dimethylaminobenzyl)-1,2,4-oxadiazolyl]carbonyl)-2-(S)-methylpropyl]-L-prolinamide; 2-(5-[(Benzyloxycarbonyl)amino]-6-oxo-2-(4-fluorophenyl)-1,6-dihydro-1-pyrimidinyl]-N-[1-(3-(5-(3-trifluoromethylbenzyl)-1,2,4-oxadiazolyl]carbonyl)-(S)-2-methylpropyl]acetamide; 2-(5-Amino-6-oxo-2-(4-fluorophenyl)-1,6-dihydro-1-pyrimidinyl]-N-[1-(3-(5-(3-trifluoromethylbenzyl)-1,2,4-oxadiazolyl]carbonyl)-2-(S)-methylpropyl]acetamide; 2-(5-[(Benzyloxycarbonyl)amino]-6-oxo-2-(4-fluorophenyl)-1,6-dihydro-1-pyrimidinyl]-N-[1-(2-(5-(3-methylbenzyl)-1,3,4-oxadiazolyl]carbonyl)-(S)-2-methylpropyl] acetamide; 2-(5-Amino-6-oxo-2-(4-fluorophenyl)-1,6-dihydro-1-pyrimidinyl]-N-[1-(2-(5-(3-methylbenzyl)-1,3,4-oxadiazolyl]carbonyl)-2-methylpropyl]acetamide; (Pyrrole-2-carbonyl)-N-(benzyl)glycyl-N-[1-(2-(5-(3-methylbenzyl)-1,3,4-oxadiazolyl]carbonyl)-2-(S)-methylpropyl]amide; (Pyrrole-2-carbonyl)-N-(benzyl)glycyl-N-[1-(3-(5-(3-trifluoromethylbenzyl) ]-1,2,4-oxadiazolyl)-(S)-methylpropyl]amide; (2S,5S)-5-Amino-1,2,4,5,6,7-hexahydroazepino-[3,2,1]-indole-4-one-carbonyl-N-[)1-(2-(5-(3-methylbenzyl)-1,3,4-oxadiazolyl]carbonyl)-(R,S)-2-methylpropyl ]amide; BTD-[1-(2-(5-(3-methylbenzyl)-1,3,4-oxadiazolyl]carbonyl)-2-(S)-methylpropylamide; (R,S)-3-Amino-2-oxo-5-phenyl-1,4,-benzodiazepine-N-[1-(2-(5-(3-methylbenzyl)-1,3,4-oxadiazolyljcarbonyl)-2-(S)-methylpropyl]acetamide; (Benzyloxycarbonyl)-L-valyl-2-L-(2,3-dihydro-1H-indole)-N-[1-(2-(5-(3-methylbenzyl)-1,2,3-oxadiazolyl]carbonyl)-2-(S)-methylpropyl]amide; (Benzyloxycarbonyl)-L-valyl-2-L-(2,3-dihydro-1H-indole)-N-[1-(3-(5-(3-trifluoromethylbenzyl)-1,2,4-oxadiazolyl]carbonyl)-2-(S)-ethylpropyl]amide; Acetyl-2-L-(2,3-dihydro-1H-indole)-N-[1-(2-(5-(3-methylbenzyl)-1,3,4-oxadiazolyl]carbonyl)-2-(S)-methylpropyl]amide; 3-(S)-(Benzyloxycarbonyl)amino)-.epsilon.-lactam-N-[1-2-(5-(3-methylbenzyl)-1,2,4-oxadiazolyl]carbonyl)-2-(S)-methylpropyl]acetamide; 3-(S)-(Amino)-.epsilon.-lactam-N-[1-(2-(5-(3-methylbenzyl)-1,3,4-oxadiazolyl]carbonyl)-2-(S)-methylpropyl]acetamide trifluoroacetic acid salt; 3-(S)-[(4-morpholinocarbonyl-butanoyl)amino]-.epsilon.-lactam-N-[1-(2-(5-(3-methylbenzyl)-1,3,4-oxadiazolyl]carbonyl)-2-(R,S)-methylpropyl]acetamide; 6-[4-Fluorophenyl]-.epsilon.-lactam-N-[1-(2-(5-(3-methylbenzyl)-1,3,4-oxadiazolyl]carbonyl)-2-(S)-methylpropyl]acetamide; 2-(2-(R,S)-Phenyl-4-oxothiazolidin-3-yl]-N-[1-(2-(5-(3-methylbenzyl)-1,3,4-oxadiazolyl]carbonyl)-2-(S)-methylpropyl]acetamide; 2-(2-(R,S)-phenyl-4-oxothiazolidin-3-yl]-N-[1-(2-(5-(3-methylbenzyl)-1,3,4-oxadiazolyl]hydroxymethyl)-2-(S)-methylpropyl]acetamide; 2-(2-(R,S)-Benzyl-4-oxothiazolidin-3-yl]-N-[1-(2-(5-(3-methylbenzyl)-1,3,4-oxadiazolyl]carbonyl)-2-(S)-methylpropyl]-acetamide; 2-(2-(R,S)-Benzyl-4-oxothiazolidin-3-yloxide]-N-[1-(3-(5-(3-trifluoromethylbenzyl)-1,2,4-oxadiazolyl]carbonyl)-2-(R,S,)-methylpropyl]acetamide; (1-Benzoyl-3,8-quinazolinedione)-N-[1-(2-(5-(3-methylbenzyl)-1,3,4-oxadiazolyljcarbonyl)-2-(S)-methylpropyl ]acetamide; (1-Benzoyl-3,6-piperazinedione)-N-[1-(2-(5-(3-methylbenzyl)-1,3,4-oxadiazolyl]carbonyl)-2-(S)-methylpropyl]acetamide; (1-Phenyl-3,6-piperazinedione)-N-[1-(2-(5-(3-methylbenzyl)-1,3,4-oxadiazolyl]carbonyl)-2-(S)-methylpropyl]acetamide; [(1-Phenyl-3,6-piperazinedione)-N-[1-(3-(5-(3-trifluoromethylbenzyl)-1,2,4--oxadiazolyl]carbonyl)]-2-(S)-methylpropyl]acetamide; 3-[(Benzyloxycarbonyl)amino]-quinolin-2-one-N-[1-(2-(5-(3-methybenzyl)-1,3,4-oxadiazolyl]carbonyl)-2-(S)-methylpropyl]acetamide; 3-[(Benzyloxycarbonyl)amino]-7-piperidinyl-quinolin-2-one-N-[1-(2-(5-(3-methybenzyl)-1,3,4-oxadiazolyl]carbonyl)-2-(S)-methylpropyl]acetamide; 3-(Carbomethoxy-quinolin-2-one-N-[1-(2-(5-(3-methybenzyl)-1,3,4-oxadiazolyl]carbonyl)-2-(S)-methylpropyl]acetamide; 3-(Amino-quinolin-2-one)-N-[1-(2-(5-(3-methylbenzyl)-1,3,4-oxadiazolyl]carbonyl)-2-(S)-methylpropyl]acetamide; 3-[(4-Morpholino)aceto]amino-quinolin-2-one-N-[1-(2-(5-(3-methylbenzyl )-1,3,4-oxadiazolyl]carbonyl)-2-(S)-methylpropyl]acetamide; 3,4-Dihydro-quinolin-2-one-N-[1-(2-(5-(3-methylbenzyl)-1,3,4-oxadiazolyl]carbonyl)-2-(S)-methylpropyl]acetamide; 1-Acetyl-3-(4-fluorobenzylidene)piperazine-2,5-dione-N-[1-(2-(5-(3-methylbenzyl)-1,3,4-oxadiazolyl]carbonyl)-2-(S)-methylpropyl]acetamide; 1-Acetyl-3-(4-dimethylaminobenzylidene)piperazine-2,5-dione-N-[1-(2-(5-(3-methylbenzyl)-1,3,4-oxadiazolyl]carbonyl)-2-(S)-methylpropyl]acetamide; 1-Acetyl-3-(4-carbomethoxybenzylidene)piperazine-2,4-dione-N-[1-(2-(5-(3-methylbenzyl)-1,3,4-oxadiazolyl]carbonyl)-2-(S)-methylpropyl]acetamide; 1-Acetyl-3-[(4-pyridyl)methylene]piperazine-2,4-dione-N-[1-(2-(5-(3-methylbenzyl)-1,3,4-oxadiazolyl]carbonyl)-2-(S)-methylpropyl]acetamide; 4-[1-Benzyl-3-(R)-benzyl-piperazine-2,5,-dione]-N-[1-(2-[-5-l -(3-methylbenzyl)-1,3,4-oxadiazolyl]carbonyl)-2-(S)-methylpropyl]acetamide; 4-[1-Benzyl-3-(S)-benzyl piperazine-2,5,-dione]-N-[1-(2-(5-(3-methylbenzyl )-1,3,4-oxadiazolyl]carbonyl)-2-(S)-methylpropyl]acetamide; 4-[ 1 -Benzyl-3(R)-benzylpiperazine-2,5,-dione]-N-[1-(3-(5-(3-trifluoromethylbenzyl)-1,2,4-oxadiazolyl]carbonyl)-2-(S)-methylpropyl]acetamide; 4-[1-Benzyl-3-(S)-benzylpiperazine-2,5,-dione]-N-[1-(3-(5-(3-trifluoromethylbenzyl)-1,2,4-oxadiazolyl]carbonyl)-2-(S)-methylpropyl]acetamide; 4-[1-Benzyl-3-(S)-benzylpiperazine-2,5,-dione]-N-[1-(3-(5-(2-dimethylaminoethyl)-1,2,4--oxadiazolyl]carbonyl)-2-(S )-methylpropyl]acetamide; 4-[1-Methyl-3-(R,S)-phenylpiperazine-2,5,-dione]-N-[1-(3-(5-(3-trifluoromethylbenzyl)-1,2,4-oxadiazolyl]carbonyl)-2-(S)-methylpropyl]acetamide; 4-3-Methyl-3-(R,S)-phenylpiperazine-2,5,-dione]-N-[1-(2-(5-(3-methylbenzyl)-1,3,4-oxadiazolyl]carbonyl)-2-(S)-methylpropyl]acetamide; 4-[1-(4-Morpholinoethyl)-3-(R)-benzylpiperazine-2,5,-dione]-N-[1-(2-(5-(3-methylbenzyl)-1,3,4-oxadiazolyl]carbonyl)-2-(S)-methylpropyl]acetamide; 5-(R,S)-Phenyl-2,4-imidazolidinedione-N-[1-(2-(5-(3-methylbenzyl)-1,3,4-oxadiazolyl]carbonyl)-2-(S)-methylpropyl]acetamide; 5-(R)-Benzyl-2,4-imidazolidinedione-N-[1-(2-(5-(3-methylbenzyl)-1,3,4-oxadiazolyl]carbonyl)-2-(S)-methylpropyl]acetamide; 5-(S)-Benzyl-2,4-imidazolidinedione-N-[1-(2-(5-(3-methylbenzyl)-1,2,4-oxadiazolyl]carbonyl)-2-(S)-methylpropyl]acetamide; 5-(S)-Benzyl-2,4-imidazolidinedione-N-[1-(3-(5-(3-trifluoromethylbenzyl)-1,2,4-oxadiazolyl]carbonyl)-2-(S)-methylpropyl]acetamide; 5-(R)-Benzyl-2,4-imidazolidinedione-N-[1-(3-(5-(3-trifluoromethylbenzyl)-1,2,4--oxadiazolyl]carbonyl)-2-(S)-methylpropyl]acetamide; 1-Benzyl-4-(R)-benzyl-2,4-imidazolidinedione-N-[1-(2-(5-(3-methylbenzyl)-1,3,4-oxadiazolyl]carbonyl)-2-(S)-methylpropyl]acetamide; and 1-Benzyl-4-(R)-benzyl-2,4-imidazolidinedione-N-[1-(3-(5-(3-trifluoromethylbenzyl)-1,2,4-oxadiazolyl]carbonyl)-2-(S)-methylpropyl]acetamide among others. Methods of making these molecules and derivatives thereof are well known in the art and can be found for example in U.S. Pat. Nos. 5,807,829; 5,891,852; 5,869,455; 5,861,380; and 5,801,148, which is incorporated herein by way of reference in its entirety.

Other small man-made molecules useful in this invention comprise phenylenedialkanoate esters, which are also effective in the mouse model. Specific examples of certain phenylenedialkanoate esters include but are not limited to: 2,2′-(1,4-phenylene)dibutyric acid; tert-butyl-3-chloro-pivaloate; dimethyl-2,2′-(1,4-phenylene)diisobutyrate; 2,2′-(1,4-phenylene)diisobutyric acid; bis(sulfoxides); Obis(sulfones); and bis(4-(2′-carboxy-2′-methylpropylsulfonyl)phenyl)2,2′-(1,4-phenylene)diisobutyrate among others. More specifically, U.S. Pat. No. 5,216,022 teaches other small molecules useful for the practice of this invention, including: Benzyloxycarbonyl-L-valyl-N-[1-(2-[-5-(3-methylbenzyl)-1,3,4-oxadiazolyl]carbonyl)-2-(S)-methylpropyl]-L-prolinamide (also known as CE-2072, Benzyloxycarbonyl-L-valyl-N-[1-(2-(3-methylbenzyl)-1,3,4-oxadiazolyl]carbonyl)-2-(S)-methylpropyl]-L-prolinamide; Benzyloxycarbonyl-L-valyl-N-[1-(2-(5-(methyl)-1,3,4-oxadiazoly]carbonyl)-2-(S)-methylpropyl]-L-prolinamide; Benzyloxycarbonyl)-L-valyl-N-[1-(2-(5-(3-trifluoromethylbenzyl)-1,3,4-oxadiazolyl]carbonyl)-2-(S)-methylpropyl]-L-prolinamide; (Benzyloxycarbonyl)-L-valyl-N-[1-(2-(5-(4-Dimethylaminobenzyl)-1,3,4-oxadiazolyl]carbonyl)-2-(S)-methylpropyl]-L-prolinamide; Benzyloxycarbonyl)-L-valyl-N-[1-(2-(5-(1-napthylenyl)-1,3,4-oxadiazolyl]carbonyl)-2-(S)-methylpropyl]-L-prolinamide; (Benzyloxycarbonyl)-L-valyl-[1-(3-(5-(3,4-methylenedioxybenzyl)-1,2,4-oxadiazolyl ]carbonyl)-2-(S)-methylpropyl]-L-prolinamide; Benzyloxycarbonyl)-L-valyl-N-[1-(3-(5-(3,5-dimethylbenzyl)-1,2,3-oxadiazolyl]carbonyl)-2-(S)-methylpropyl]-L-prolinamide; (Benzyloxycarbonyl)-L-valyl-N-[1-(3-(5-(3,5-dimethoxybenzyl)-1,2,4-oxadiazolyl]carbonyl)-2-(S)-methylpropyl]-L-prolinamide; (Benzyloxycarbonyl)-L-valyl-N-[1-(3-(5-(3,5-ditrifluoromethylbenzyl)-1,2,3-oxadiazolyl]carbonyl)-2-(S)-methylpropyl]-L-prolinamide; (Benzyloxycarbonyl)-L-valyl-N-[1-(3-(5-(3-methylbenzyl)-1,2,4-oxadiazolyl]carbonyl)-2-(S)-methylpropyl]-L-prolinamide; (Benzyloxycarbonyl)-L-valyl-N-[1-(3-(5-(biphenylmethine)-1,2,3--oxadiazolyl]carbonyl)-2-(S)-methylpropyl]-L-prolinamide; (Benzyloxycarbonyl)-L-valyl-N-[1-(3-(5-(4-phenylbenzyl)-1,2,4-oxadiazolyl]carbonyl)-2-(S)-methylpropyl]-L-prolinamide; (Benzyloxycarbonyl)-L-valyl-N-[1-(3-(5-(3-phenylbenzyl)-1,2,4-oxadiazolyl]carbonyl)-2-(S)-methylpropyl]-L-prolinamide; (Benzyloxycarbonyl)-L-valyl-N-[1-(3-(5-(3-phenoxybenzyl)-1,2,4--oxadiazolyl]carbonyl)-2-(S)-methylpropyl]-L-prolinamide; (Benzyloxycarbonyl)-L-valyl-N-[1-(3-(5-(cyclohexylmethylene)-1,2,4-oxadiazolyl]carbonyl)-2-(S)-methylpropyl]-L-prolinamide; (Benzyloxycarbonyl)-L-valyl-N-[1-(3-(5-(3-trifluoromethyldimethylmethylene)-1,2,4-oxadiazolyl]carbonyl)-2-(S)-methylpropyl]-L-prolinamide; (Benzyloxycarbonyl)-L-valyl-N-[1-(3-(5-(1-napthylmethylene)-1,2,4-oxadiazolyl]carbonyl)-2-(S)-methylpropyl]-L-prolinamide; (Benzyloxycarbonyl)-L-valyl-N-[1-(3-(5-(3-pyridylmethyl)-1,2,4-oxadiazolyl]carbonyl)-2-(S )-methylpropyl]-L-prolinamide; (Benzyloxycarbonyl)-L-valyl-N-[1-(3-(5-(3,5-diphenylbenzyl)-1,2,4-oxadiazolyl]carbonyl)-2-(S)-methylpropyl]-L-prolinamide; (Benzyloxycarbonyl)-L-valyl-N-[1-(3-(5-(4-dimethylaminobenzyl)-1,2,4-oxadiazolyl]carbonyl)-2-(S)-methylpropyl]-L-prolinamide; 2-(5-[(Benzyloxycarbonyl)amino]-6-oxo-2-(4-fluorophenyl)-1,6-dihydro-1-pyrimidinyl]-N-[1-(3-(5-(3-trifluoromethylbenzyl)-1,2,4-oxadiazolyl]carbonyl)-(S)-2-methylpropyl]acetamide; 2-(5-Amino-6-oxo-2-(4-fluorophenyl)-1,6-dihydro-1-pyrimidinyl]-N-[1-(3-(5-(3-trifluoromethylbenzyl)-1,2,4-oxadiazolyl]carbonyl)-2-(S)-methylpropyl]acetamide; 2-(5-[(Benzyloxycarbonyl)amino]-6-oxo-2-(4-fluorophenyl)-1,6-dihydro-1-pyrimidinyl]-N-[1-(2-(5-(3-methylbenzyl)-1,3,4-oxadiazolyl]carbonyl)-(S)-2-methylpropyl]acetamide; 2-(5-Amino-6-oxo-2-(4-fluorophenyl)-1,6-dihydro-1-pyrimidinyl]-N-[1-(2-(5-(3-methylbenzyl)-1,3,4-oxadiazolyl]carbonyl)-2-methylpropyl]acetamide; (Pyrrole-2-carbonyl)-N-(benzyl)glycyl-N-[1-(2-(5-(3-methylbenzyl)-1,3,4-oxadiazolyl]carbonyl)-2-(S)-methylpropyl]amide; (Pyrrole-2-carbonyl)-N-(benzyl)glycyl-N-[1-(3-(5-(3-trifluoromethylbenzyl)]-1,2,3-oxadiazolyl)-(S)-methylpropyl]amide; (2S,5S)-5-Amino-1,2,4,5,6,7-hexahydroazepino-[3,2,1 ]-indole-4-one-carbonyl-N-[ 1 -(2-(5-(3-methylbenzyl)-1,3,4-oxadiazolyl]carbonyl)-(R,S)-2-methylpropyl]amide; BTD-[1-(2-(5-(3-methylbenzyl)-1,3,4-oxadiazolyl]carbonyl)-2-(S)-methylpropyl]amide; (R,S)-3-Amino-2-oxo--5-phenyl-1,4,-benzodiazepine-N-[1-(2-(5-(3-methylbenzyl)-1,3,4-oxadiazolyl]carbonyl)-2-(S)-methylpropyl]acetamide; (Benzyloxycarbonyl)-L-valyl-2-L-(2,3-dihydro-1H-indole)-N-[1-(2-(5-(3-methylbenzyl)-1,3,4-oxadiazolyl]carbonyl)-2-(S)-methylpropyl]amide; (Benzyloxycarbonyl)-L-valyl-2-L-(2,3-dihydro-1H-indole)-N-[1-(3-(5-(3-trifluoromethylbenzyl)-1,2,4-oxadiazolyl]carbonyl)-2-(S)-methylpropyl]amide; Acetyl-2-L-(2,3-dihydro-1H-indole)-N-[1-(2-(5-(3-methylbenzyl)-1,3,4-oxadiazolyl]carbonyl)-2-(S)-methylpropyl]amide; 3-(S)-(Benzyloxycarbonyl)amino)-ε-lactam-N-[1-(2-(5-(3-methylbenzyl)-1,3,4-oxadiazolyl]carbonyl)-2-(S)-methylpropyl]acetamide; 3-(S)-(Amino)-ε-lactam-N-[1-(2-(5-(3-methyl benzyl)-1,3,4-oxadiazolyl]carbonyl )-2-(S)-methylpropyl]acetamide trifluoroacetic acid salt; 3-(S)-[(4-morpholinocarbonyl-butanoyl)amino]-ε-lactam-N-[1-(2-(5-(3-methylbenzyl)-1,3,4-oxadiazolyl]carbonyl)-2-(R,S )-methylpropyl ]acetamide; 6-[4-Fluorophenyl]-ε-lactam-N-[1-(2-(5-(3-methylbenzyl)-1,3,4-oxadiazolyl]carbonyl)-2-(S)-methylpropyl]acetamide; 2-(2-(R,S)-Phenyl-4-oxothiazolidin-3-yl]-N-[1-(2-(5-(3-methylbenzyl)-1,2,3-oxadiazolyl]carbonyl)-2-(S)-methylpropyl]acetamide; 2-(2-(R,S)-phenyl-4--oxothiazolidin-3-yl]-N-[1-(2-(5-(3-methylbenzyl)-1,3,4-oxadiazolyl]hydroxymethyl)-2-(S)-methylpropyl]acetamide; 2-(2-(R,S)-Benzyl-4-oxothiazolidin-3-yl]-N-[1-(2-(5-(3-methylbenzyl)-1,3,4-oxadiazolyl]carbonyl)-2-(S)-methylpropyl]-acetamide; 2-(2-(R,S)-Benzyl-4-oxothiazolidin-3-yl oxide]-N-[1-3-(5-(3-trifluoromethylbenzyl)-1,2,4-oxadiazolyl]carbonyl)-2-(R,S,)-methylpropyl]acetamide; (1-Benzoyl-3,8-quinazolinedione)-N-[1-(2-(5-(3-methylbenzyl)-1,3,4-oxadiazolyl]carbonyl)-2-(S)-methylpropyl]acetamide; (1-Benzoyl-3,6-piperazinedione)-N-[1-(2-(5-(3-methylbenzyl)-1,3,4-oxadiazolyl]carbonyl)-2-(S)-methylpropyl]acetamide; (1-Phenyl-3,6-piperazinedione)-N-[1-(2-(5-(3-methylbenzyl)-1,3,4-oxadiazolyl]carbonyl)-2-(S)-methylpropyl]acetamide; [(1-Phenyl-3,6-perazinedione)-N-[1-(3-(5-(3-trifluoromethylbenzyl)-1,2,4--oxadiazolyl]carbonyl)]-2-(S)-methylpropyl]acetamide; 3-[(Benzyloxycarbonyl)amino]-quinolin-2-one-N-[1-(2-(5-(3-methylbenzyl)-1,3,4-oxadiazolyl]carbonyl)-2-(S)-methylpropyl]acetamide; 3-[(Benzyloxycarbonyl)amino]-7-piperidinyl-quinolin-2-one-N-[1-(2-(5-(3-methylbenzyl)-1,3,4-oxadiazolyl]carbonyl)-2-(S)-methylpropyl]acetamide; 3-(Carbomethoxy-quinolin-2-one-N-[1-(2-(5-(3-methybenzyl)-1,3,4-oxadiazolyl]carbonyl)-2-(S)-methylpropyl]acetamide; 3-(Amino-quinolin-2-one)-N-[1-(2-(5-(3-methylbenzy)-1,3,4-oxadiazolyl]carbonyl)-2-(S)-methylpropyl]acetamide; 3-[(4-Morpholino)aceto]amino-quinolin-2-one-N-[1-(2-(5-(3-methylbenzyl)-1,3,4-oxadiazolyl]carbonyl)-2-(S)-methylpropyl]acetamide; 3,4-Dihydro-quinolin-2-one-N-[1-(2-(5-(3-methylbenzyl)-1,3,4-oxadiazolyl]carbonyl)-2-(S)-methylpropyl]acetamide; 1-Acetyl-3-(4-fluorobenzylidene) piperazine-2,5-dione-N-[1-(2-(5-(3-methylbenzyl)-1,3,4-oxadiazolyl]carbonyl)-2-(S)-methylpropyl]acetamide; 1-Acetyl-3-(4-dimethylaminobenzylidene)piperazine-2,4-dione-N-[1-(2-(5-(3-methylbenzyl)-1,3,4-oxadiazolyl]carbonyl)-2-(S)-methylpropyl]acetamide; 1-Acetyl-3-(4-carbomethoxybenzylidene)piperazine-2,4-dione-N-[1-(2-(5-(3-methylbenzyl)-1,3,4-oxadiazolyl]carbonyl)-2-(S)-methylpropyl]acetamide; 1-Acetyl-3-[(4-pyridyl)methylene]piperazine-2,4-dione-N-[1-(2-(5-(3-methylbenzyl)-1,3,4-oxadiazolyl]carbonyl)-2-(S)-methylpropyl]acetamide; 4-[1-Benzyl-3-(R)-benzyl-piperazine-2,5,-dione]-N-[1-(2-[5-(3-methylbenzyl)-1,3,4-oxadiazolyljcarbonyl)-2-(S)-methylpropyl]acetamide; 4-[1-Benzyl-3-(S)-benzyl piperazine-2,5,-dione]-N-[1-(2-(5-(3-methylbenzyl)-1,2,3-oxadiazolyljcarbonyl)-2-(S)-methylpropyl]acetamide; 4-[1-Benzyl-3(R)-benzylpiperazine-2,5,-dione]-N-[1-(3-(5-(3-trifluoromethylbenzyl)-1,2,4-oxadiazolyl]carbonyl)-2-(S)-methylpropyl]acetamide; 4-[1-Benzyl-3-(S)-benzylpiperazine-2,5,-dione]-N-[1-(3-(5-(3-trifluoromethylbenzyl)-1,2,4-oxadiazolyl]carbonyl)-2-(S)-methylpropyl]acetamide; 4-[1-Benzyl-3-(S)-benzyl piperazine-2,5,-dione]-N-[1-(3-(5-(2-dimethylaminoethyl)-1,2,4-oxadiazolyljcarbonyl)-2-(S)-methylpropyl]acetamide; 4-[1-Methyl-3-(R,S)-phenylpiperazine-2,5,-dione]-N-[1-(3-(5-(3-trifluoromethylbenzyl)-1,2,3--oxadiazolyl]carbonyl)-2-(S)-methylpropyl]acetamide; 4-[[-Methyl-3-(R,S)-phenylpiperazine-2,5,-dione]-N-[1-(2-(5-(3-methylbenzyl)-1,3,4-oxadiazolyl]carbonyl)-2-(S)-methylpropyl]acetamide; 4-[1-(4-Morpholinoethyl)3-(R)-benzylpiperazine-2,5,-dione]-N-[1-(2-(5-(3-methylbenzyl)-1,3,4-oxadiazolyl]carbonyl)-2-(S)-methylpropyl]acetamide; 5-(R,S)-Phenyl-2,4-imidazolidinedione-N-[1-(2-(5-(3-methylbenzyl)-1,3,4-oxadiazolyl]carbonyl)-2-(S)-methylpropyl]acetamide; 5-(R)-Benzyl-2,4-imidazolidinedione-N-[1-(2-(5-(3-methylbenzyl)-1,3,4-oxadiazolyljcarbonyl)-2-(S)-methylpropyl]acetamide; 5-(S)-Benzyl-2,4-imidazolidinedione-N-[1-(2-(5-(3-methylbenzyl)-1,3,4-oxadiazolyl]carbonyl)-2-(S)-methylpropyl]acetamide; 5-(S)-Benzyl-2,4-imidazolidinedione-N-[1-(3-(5-(3-trifluoromethylbenzyl)-1,2,4-oxadiazolyl]carbonyl)-2-(S)-methylpropyl]acetamide; 5-(R)-Benzyl-2,4-imidazolidinedione-N-[1-(3-(5-(3-trifluoromethylbenzyl)-1,2,3--oxadiazolyljcarbonyl)-2-(S)-methylpropyl]acetamide; 1-Benzyl-4-(R)-benzyl-2,4-imidazolidinedione-N-[1-(2-(5-(3-methylbenzyl)-1,3,4-oxadiazolyl]carbonyl)-2-(S)-methylpropyl] acetamide; and 1-Benzyl-4--(R)-benzyl-2,4-imidazolidinedione-N-[1 -(3-(5-(3-trifluoromethylbenzyl)-1,2,4-oxadiazolyl]carbonyl)-2-(S)-methylpropyl]acetamide among others.

Methods of making these molecules and derivatives thereof are well known in the art and can be found in aforementioned U.S. Pat. No. 5,216,022, which is incorporated herein by way of reference in its entirety.

Likewise, U.S. Pat. No. 5,869,455 discloses N-substituted derivatives; U.S. Pat. No. 5,861,380 protease inhibitors-keto and di-keto containing ring systems; U.S. Pat. No. 5,807,829 serine protease inhibitor--tripeptoid analogues; U.S. Pat. No. 5,801,148 serine protease inhibitors-proline analogues; U.S. Pat. No. 5,618,792 substituted heterocyclic compounds useful as inhibitors of serine proteases. These patents and PCT publications and others as listed infra are enclosed herein by reference. Other equally advantageous molecules, which may be used instead of α₁-antitrypsin or in combination with α₁-antitrypsin are contemplated such as in WO 98/20034 disclosing serine protease inhibitors from fleas. Without limiting to this single reference one skilled in the art can easily and without undue experimentation adopt compounds such as in WO98/23565 which discloses aminoguanidine and alkoxyguanidine compounds useful for inhibiting serine proteases; WO98/50342 discloses bis-aminomethylcarbonyl compounds useful for treating cysteine and serine protease disorders; WO98/50420 cyclic and other amino acid derivatives useful for thrombin-related diseases; WO 97/21690 D-amino acid containing derivatives; WO 97/10231 ketomethylene group-containing inhibitors of serine and cysteine proteases; WO 97/03679 phosphorous containing inhibitors of serine and cysteine proteases; WO 98/21186 benzothiazo and related heterocyclic inhibitors of serine proteases; WO 98/22619 discloses a combination of inhibitors binding to P site of serine proteases with chelating site of divalent cations; WO 98/22098 a composition which inhibits conversion of pro-enzyme CPP32 subfamily including caspase 3 (CPP32/Yama/Apopain); WO 97/48706 pyrrolo-pyrazine-diones; WO 97/33996 human placental bikunin (recombinant) as serine protease inhibitor; WO 98/46597 complex amino acid containing molecule for treating viral infections and conditions disclosed hereinabove.

Other compounds having serine protease inhibitory activity are equally suitable and effective including but not limited to tetrazole derivatives as disclosed in WO 97/24339; guanidinobenzoic acid derivatives as disclosed in WO 97/37969 and in a number of U.S. Pat. Nos. 4,283,418; 4,843,094; 4,310,533; 4,283,418; 4,224,342; 4,021,472; 5,376,655; 5,247,084; and 5,077,428; phenylsulfonylamide derivatives represented by general formula in WO 97/45402; novel sulfide, sulfoxide and sulfone derivatives represented by general formula in WO 97/49679; novel amidino derivatives represented by general formula in WO 99/41231; other amidinophenol derivatives as disclosed in U.S. Pat. Nos. 5,432,178; 5,622,984; 5,614,555; 5,514,713; 5,110,602; 5,004,612; and 4,889,723 among many others.

In summary, the Examples recited hereinabove show that compounds exhibiting AAT activity such as AAT, peptides derived analogous or homologous to C-terminal end of AAT, and man-made synthetic molecules mimicking AAT action, display herpes virus-suppressive effects in vitro and in vivo.

Throughout this application various publications and patents are referenced. The disclosures of these publications and patents in their entireties are hereby incorporated by reference into this application in order to more fully describe the state of the art to which this invention pertains.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as can be applied to the essential features hereinbefore set forth, and as follows in the scope of the appended claims.

What is claimed is:

1. A method of treating a subject suffering from a herpes virus infection or a disease arising from a herpes virus infection comprising:
administering to the subject a therapeutically effective amount of a substance exhibiting mammalian alpha-1-antitrypsin (AAT) or AAT-like activity.

2. The method of claim 1 in which said disease is malaise, fever, chills, rhinitis, diarrhea, atopic eczema, encephalitis, keratoconjunctivitis, pharyngitis, gingivostomatitis, herpetic hepatitis, recurrent orofacial mucocutaneous lesions or herpes labialis, chicken pox skin sores, erythema multiforme, idiopathic burning mouth, aphthous ulceration, Behcet's syndrome, or combinations thereof.

3. The method of claim 1 in which said disease is mononucleosis, Burkitt's lymphoma, primary effusion lymphomas, multiple myeloma, angioimmunoblastic lymphadenopathy, Castleman's disease, acquired immune deficiency syndrome (AIDS)-related lymphoma, post-transplantation lymphoproliferative disease, Hodgkin's disease, T-cell lymphomas, oral hairy leukoplakia, lymphoproliferative disease, lymphoepithelial carcinoma, body-cavity-based lymphoma or B-cell lymphomas, non-keratinising carcinoma, squamous cell nasopharyngeal carcinoma, kidney transplant-associated epithelial tumors, malignant mesothelioma, angiosarcoma, Kaposi's sarcoma, angiolymphoid hyperplasia, prostatic neoplasm, cervical cancer, neoplasms of the vulva, retinoblastoma, Li-Fraumeni syndrome, Gardner's syndrome, Werner's syndrome, nervoid basal cell carcinoma syndrome, neurofibromatosis type 1, or combinations thereof.

4. The method of claim 1 in which said disease is polyneuropathy, motor neuropathy, sensory neuronopathy, polyradiculoneuropathy, autonomic neuropathy, focal or multifocal cranial neuropathy, radiculopathy, plexopathy resulting from tumor infiltration, or combinations thereof.

5. The method of claim 1 in which the substance comprises AAT.

6. The method of claim 5 in which the AAT is substantially purified from a wild type, mutant, or transgenic mammalian source.

7. The method of claim 5 in which the AAT is isolated from a culture of wild type, mutant, or transformed cells.

8. The method of claim 1 in which the herpes virus comprises a virus selected from the group consisting of herpes simplex virus type I (HSV-1), herpes simplex virus type II (HSV-2), cytomegalovirus (CMV), Epstein-Barr virus (EBV), varicella zoster virus (VZV), herpes zoster virus, human herpes virus type V (HHV-5), human herpes virus type VI (HHV-6), human herpes virus type VIII (HHV-8), and combinations thereof.

9. The method of claim 1 in which the substance comprises a compound selected from substituted oxadiazole, thiadiazole, triazole peptoids, phenylenedialkanoate esters, tetrazole, guanidinobenzoic acid, phenylsulfonylamide, sulfide, sulfoxide, sulfone amidino, amidinophenol, or derivatives thereof.

10. The method of claim 1 in which the substance comprises a peptide of the general formula: N_{T}—X₁—X₂—X₃—X₄—X₅—C_{T} or a physiologically acceptable salt thereof, in which N_{T} comprises an amino acid residue positioned at the peptide's N-terminal end, including C, an acetyl group, or a succinyl group, provided that N_{T} can also be absent; X₁ comprises an amino acid residue, including F or A; X₂ comprises an amino acid residue, including C, V, L, M, I, A, C, or S; X₃ comprises an amino acid residue, including F, A, V, M, L, I, Y, or C; X₄ comprises an amino acid residue, including L, A, F, I, V, M, C, G, or S; X₅ comprises an amino acid residue, including M, A, I, L, V, F, or G; and CT comprises an amino acid residue positioned at the peptide's C-terminal end, including C, an amide group, a substituted amide group, or an ester group, provided that CT can also be absent, and in which the amino acid residue can be either an L- or a D-stereoisomeric configuration.

11. The method of claim 1 in which the therapeutically effective amount of the substance exhibiting mammalian alpha-1-antitrypsin (AAT) or AAT-like activity is in the range of about 1 μg per kg to about 100 mg per kg of body weight of the mammalian subject.

12. The method of claim 1 in which the therapeutically effective amount of the substance is administered systemically or topically.

13. The method of claim 1 in which said herpes virus infection is one of a mucosa and is selected from an infection of the oral soft tissues, middle ear, gastrointestinal tract, urogenital tract, airway/lung tissue, eye, peritoneal membranes, or combinations thereof.

14. The method of claim 13 in which the substance is administered topically to said mucosa.

15. The method of claim 1 in which the substance is (benzyloxycarbonyl)-L-valyl-N-[1-(3-(5-(3-trifluoromethylbenzyl)-1,2,4-oxadiazolyl)carbonyl)-2-(S)- methylpropyl]-L-prolinamide benzyloxycarbonyl)-L-valyl-N-[1-(3-(5-(2-phenylethyl)-1,2,4-oxadiazolyl)carbonyl)-2-(S)-methylpropyl]-L-prolinamide; (benzyloxycarbonyl)-L-valyl-N-[1-(3-(5-(2-methoxybenzyl)-1,2,4-oxadiazolyl)carbonyl)-2-(S )-methylpropyl]-L-prolinamide; (benzyloxycarbonyl)-L-valyl-N-[1-(3-(5-(trifluoromethyl)-1,2,4-oxadiazolyl)carbonyl)-2-(S)-methylpropyl]-L-prolinamide; (benzyloxycarbonyl)-L-valyl-N-[1-(3-(5-(methyl)-1,2,4-oxadiazolyl)carbonyl)-2-(S)-Methylpropyl]-L-Prolinamide; (Benzyloxycarbonyl)-L-Valyl-N-[1-(3-(5-(difluoromethyl)-1,2,4-oxadiazolyl)carbonyl)-2-(S)-Methylpropyl]-L-Prolinamide; (Benzyloxycarbonyl)-L-Valyl-N-[1-(3-(5-(benzyl)-1,2,4-oxadiazolyl)carbonyl)-2-(S)-Methylpropyl]-L-Prolinamide; (Benzyloxycarbonyl)-L-Valyl-N-[1-(3-(5-(3-methoxybenzyl)-1,2,3-oxadiazolyl)carbonyl)-2-(S)-Methylpropyl]-L-Prolinamide; (Benzyloxycarbonyl)-L-Valyl-N-[1-(3-(5-(2,6-difluorobenzyl)-1,2,4-oxadiazolyl)carbonyl)-2-(S)-Methylpropyl]-L-Prolinamide; (Benzyloxycarbonyl)-L-Valyl-N-[1-(3-(5-(trans-styryl)-1,2,4-oxadiazolyl)carbonyl)-2-(S)-Methylpropyl]-L-Prolinamide; (Benzyloxycarbonyl)-L-Valyl-N-[1-(3-(5-(trans-4-Trifluoro methylstyryl)-1,2,4-oxadiazolyl)carbonyl)-2-(S)-Methylpropyl]-L-Prolinamide; (Benzyloxycarbonyl)-L-Valyl-N-[1-(3-(5-(trans-4--Methoxystyryl)-1,2,4-oxadiazolyl)carbonyl)-2-(S)-Methylpropyl]-L-Prolinamide; (Benzyloxycarbonyl)-L-Valyl-N-[1-(3-(5-(3-Thienylmethyl)-1,2,4-oxadiazolyl)carbonyl)-2-(S)-Methylpropyl]-L-Prolinamide; (Benzyloxycarbonyl)-L-Valyl-N-[1-(3-(5-(Phenyl)-1,2,4-oxadiazolyl)carbonyl )-2-(S)-methylpropyl]-L-prolinamide; and (Benzyloxycarbonyl)-L-Valyl-N-[1-(b 3-(b 5-(3-Phenylpropyl)-1,2,4-oxadiazolyl)carbonyl)-2-(S)-Methylpropyl]-L-Prolinamide, Benzyloxycarbonyl-L-valyl-N-[1-(2-[5-(3-methylbenzyl)-1,3,4-oxadiazolyl]carbonyl)-2-(S)-methylpropyl]-L-prolinamide, Benzyloxycarbonyl-L-valyl-N-[1-(2-(3-methylbenzyl )-1,3,4-oxadiazolyl]carbonyl)-2-(S)-methylpropyl]-L-prolinamide; Benzyloxycarbonyl-L-valyl-N-[1-(2-(5-(methyl)-1,3,4-oxadiazoly]carbonyl)-2-(S)-methylpropyl]-L-prolinamide; Benzyloxycarbonyl)-L-valyl-N-[1-(2-(5-(3-trifluoromethylbenzyl)-1,3,4-oxadiazolyl]carbonyl)-2-(S)-methylpropyl]-L-prolinamide; (Benzyloxycarbonyl)-L-valyl-N-[1-(2-(5-(4-Dimethylamino benzyl)-1,3,4-oxadiazolyl]carbonyl)-2-(S)-methylpropyl]-L-prolinamide; Benzyloxycarbonyl)-L-valyl-N-[1-(2-(5-(1-napthylenyl)-1,3,4-oxadiazolyl]carbonyl)-2-(S)-methylpropyl]-L-prolinamide; (Benzyloxycarbonyl)-L-valyl-[1-(3-(5-(3,4-methylenedioxybenzyl)-1,2,4-oxadiazolyl]carbonyl)-2-(S)-methylpropyl]-L-prolinamide; Benzyloxycarbonyl)-L-valyl-N-[1-(3-(5-(3,5-dimethylbenzyl)-1,2,4-oxadiazolyl]carbonyl)-2-(S)-methylpropyl]-L-prolinamide; (Benzyloxycarbonyl)-L-valyl-N-[1-(3-(5-(3,5-dimethoxybenzyl)-1,2,4-oxadiazolyl]carbonyl)-2-(S)-methylpropyl]-L-prolinamide; (Benzyloxycarbonyl)-L-valyl-N-[1-(3-(5-(3,5-ditrifluoromethylbenzyl)-1,2,4-oxadiazolyl]carbonyl)-2-(S)-methylpropyl]-L-prolinamide; (Benzyloxycarbonyl)-L-valyl-N-[1-(3-(5-(3-methylbenzyl)-1,2,4-oxadiazolyl]carbonyl)-2-(S)-methylpropyl]-L-prolinamide; (Benzyloxycarbonyl )-L-valyl-N-[1-(3-(5-(biphenylmethine)-1,2,3--oxadiazolyl]carbonyl)-2-(S)-methylpropyl]-L-prolinamide; (Benzyloxycarbonyl)-L-valyl-N-[1-(3-(5-(3-phenylbenzyl)-1,2,4-oxadiazolyl]carbonyl)-2-(S)-methylpropyl]-L-prolinamide; (Benzyloxycarbonyl)-L-valyl-N-[1-(3-(5-(3-phenylbenzyl)-1,2,4-oxadiazolyl]carbonyl)-2-(S-methylpropyl]-L-prolinamide; (Benzyloxycarbonyl)-L-valyl-N-[1-(3-(5-(3-phenoxybenzyl)-1,2,4-oxadiazolyl ]carbonyl)-2-(S)-methylpropyl]-L-prolinamide; (Benzyloxycarbonyl)-L-valyl-N-[1-(3-(5-(cyclohexylmethylene)-1,2,4-oxadiazolyl]carbonyl)-2-(S)-methylpropyl]-L-prolinamide; (Benzyloxycarbonyl)-L-valyl-N-[1-(3-(5-(3-trifluoromethyldimethylmethylene)-1,2,4-oxadiazolyl]carbonyl)-2-(S)-methylpropyl]-L-prolinamide; (Benzyloxycarbonyl)-L-valyl-N-[1-(3-(5-(1-napthylmethylene)-1,2,4-oxadiazolyl]carbonyl)-2-(S)-methylpropyl]-L-prolinamide; (Benzyloxycarbonyl)-L-valyl-N-[1-(3-(5-(3-pyridylmethyl)-1,2,4--oxadiazolyl]carbonyl)-2-(S)-methylpropyl ]-L-prolinamide; (Benzyloxycarbonyl)-L-valyl-N-[1-(3-(5-(3,5-diphenylbenzyl)-1,2,4-oxadiazolyl]carbonyl)-2-(S)-methylpropyl]-L-prolinamide; (Benzyloxycarbonyl)-L-valyl-N-[1-(3-(5-(4-dimethylaminobenzyl)-1,2,4-oxadiazolyl]carbonyl)-2-(S)-methylpropyl]-L-prolinamide; 2-(5-[(Benzyloxycarbonyl)amino]-6-oxo-2-(4-fluorophenyl)-1,6-dihydro-1-pyrimidinyl]-N-[1-(3-(5-(3-trifluoromethylbenzyl)-1,2,4-oxadiazolyl]carbonyl)-(S)-2-methylpropyl]acetamide; 2-(5-Amino-6-oxo-2-(4-fluorophenyl)-1,6-dihydro-1-pyrimidinyl]-N-[1-(3-(5-(3-trifluoromethylbenzyl)-1,3,4--oxadiazolyl]carbonyl)-2-(S)-methylpropyl]acetamide; 2-(5-[(Benzyloxycarbonyl)amino]-6-oxo-2-(4-fluorophenyl)-1,6-dihydro-1-pyrimidinyl]-N-[1-(2-(5-(3-methylbenzyl)-1,3,4-oxadiazolyl]carbonyl)-2-methylpropyl]acetamide; (Pyrrole-2-carbonyl)-N-(benzyl)glycyl-N-[1-(2-(5-(3-methylbenzyl)-1,3,4-oxadiazolyl]carbonyl)-2-(S)-methylpropyl]amide; (Pyrrole-2-carbonyl)-N-(benzyl)glycyl-N-[1-(3-(5-(3-trifluoromethylbenzyl)]-1,2,4-oxadiazolyl)-(S)-methylpropyl]amide; (Pyrrole-2-carbonyl)-N-benzyl)glycyl-N-[1-(3-(5-(3-trifluoromethylbenzyl)]-1,2,4-oxadiazolyl)-(S)-methylpropyl]amide; (2S,5S)-5-Amino-1,2,4,5,6,7-hexahydroazepino-[3,2,1]-indole-4-one-carbonyl -N-[1-(2-(5-(3-methylbenzyl)-1,3,4-oxadiazolyl]carbonyl)-(R,S)-2-methylpropyl]amide; BTD-[1-(2-(5-(3-methylbenzyl)-1,3,4-oxadiazolyl]carbonyl)-2-(S)-methylpropyl]amide; (R,S)-3-Amino-2-oxo-5-phenyl-1,4,-benzodiazepine-N-[1-(2-(5-(3-methylbenzy 1)-1,3,4-oxadiazolyl]carbonyl)-2-(S)-methylpropyl]acetamide; (Benzyloxycarbonyl)-L-valyl-2-L-(2,3-dihydro-1H-indole)-N-[1-(2-(5-(3-methylbenzyl)-1,3,4-oxadiazolyl]carbonyl )-2-(S)-methylpropyl]amide; (Benzyloxycarbonyl)-L-valyl-2-L-(2,3-dihydro-1H-indole)-N-[1-(3-(5-(3-trifluoromethylbenzyl)-1,2,4-oxadiazolyl]carbonyl )-2-(S)-methylpropyl]amide; Acetyl-2-L-(2,3-dihydro-1H-indole)-N-[1-(2-(5-(3-methylbenzyl)-1,3,4-oxadiazolyl]carbonyl)-2-(S)-methylpropyl]amide; 3-(S)-(Benzyloxycarbonyl)amino)-ε-lactam-N-[1-(2-(5-(3-methylbenzy 1)-1,3,4-oxadiazolyl]carbonyl)-2-(S)-methylpropyl]acetamide; 3-(S)-(Amino)-ε-lactam-N-[1-(2-(5-(3-methylbenzyl)-1,3,4-oxadiazolyl]carbonyl)-2-(S)-methylpropyl]acetamide trifluoroacetic acid salt; 3-(S)-[(4-morpholino carbonyl-butanoyl)amino]-ε-lactam-N-[1-(2-(5-(3-methylbenzyl)-1,3,4-oxadiazolyl]carbonyl)-2-(R,S)-methylpropyl]acetamide; 6-[4-Fluorophenyl]-ε-lactam-N-[1-(2-(5-(3-methylbenzyl)-1,3,4-oxadiazolyl]carbonyl)-2-(S)-methylpropyl]acetamide; 2-(2-(R,S)-Phenyl-4-oxothiazolidin-3-yl]-N-[1-(2-(5-(3-methylbenzyl)-1,3,4-oxadiazolyl]carbonyl)-2-(S)-methylpropyl]acetamide; 2-(2-(R,S)-phenyl-4-oxothiazolidin-3-yl]-N-[1-(2-(5-(3-methylbenzyl)-1,3,4-oxadiazolyl]hydroxymethyl)-2-(S)-methylpropyl]acetamide; 2-(2-(R,S)-Benzyl-4-oxothiazolidin-3-yl]-N-[1-(2-(5-(3-methylbenzyl)-1,3,4-oxadiazolyl]carbonyl)-2-(S)-methylpropyl]-acetamide; 2-(2-(R,S)-Benzyl-4-oxothiazolidin-3-yl oxide]-N-[1-(3-(5-(3-trifluoromethylbenzyl)-1,2,4-oxadiazolyl]carbonyl)-2-(R,S,)-methylpropyl]acetamide; (1--Benzoyl-3,8-quinazolinedione)-N-[1-(2-(5-(3-methylbenzyl)-1,3,4-oxadiazolyl]carbonyl)-2-(S)-methylpropyl]acetamide; (1-Benzoyl-3,6-piperazinedione)-N-[1-(2-(5-(3-methylbenzyl)-1,3,4-oxadiazolyl]carbonyl)-2-(S)-methylpropyl]acetamide; (1-Phenyl-3,6-piperazinedione)-N-[1-(2-(5-(3-methylbenzyl)-1,3,4-oxadiazolyl]carbonyl)-2-(S)-methylpropyl]acetamide; [(1-Phenyl-3,6-piperazinedione)-N-[1-(3-(5-(3-trifluoromethylbenzyl)-1,2,4-oxadiazolyl]carbonyl))-2-(S)-methylpropyl]acetamide; 3-[(Benzyloxycarbonyl)amino]-quinolin-2-one-N-[1-(2-(5-(3-methylbenzyl)-1,3,4-oxadiazolyl]carbonyl)-2-(S)-methylpropyl]acetamide; 3-[(Benzyloxycarbonyl)amino]-7-piperidinyl-quinolin-2-one-N-[1-(2-(5-(3-methylbenzyl)-1,3,4-oxadiazolyl]carbonyl)-2-(S)-methylpropyl]acetamide; 3-(Carbomethoxy-quinolin-2-one-N-[1-(2-(5-(3-methybenzyl)-1,3,4-oxadiazoly l]carbonyl)-2-(S)-methylpropyl]acetamide; 3-(Amino-quinolin-2-one)-N-[1-(2-(5-(3-methylbenzyl)-1,3,4-oxadiazolyl]carbonyl)-2-(S)-methylpropyl]acetamide; 3-[(4-Morpholino)aceto]amino-quinolin-2-one-N-[1-(2-(5-(3-methylbenzyl)-1,3,4-oxadiazolyl]carbonyl)-2-(S)-methylpropyl]acetamide; 3,4-Dihydro-quinolin-2-one-N-[1-(2-(5-(3-methylbenzyl)-1,3,4-oxadiazolyl]carbonyl)-2-(S)-methylpropyl]acetamide; 1-Acetyl-3-(4-fluorobenzylidene) piperazine-2,4-dione-N-[1-(2-(5-(3-methylbenzyl)-1,3,4-oxadiazolyl]carbonyl)-2-(S)-methylpropyl]acetamide; 1-Acetyl-3-(4-dimethylamino benzylidene)piperazine-2,5-dione-N-[1-(2-(5-(3-methylbenzyl)-1,3,4-oxadiazolyl]carbonyl)-2-(S)-methylpropyl]acetamide; 1-Acetyl-3-(4-carbomethoxy benzylidene)piperazine-2,5-dione-N-[1-(2-(5-(3-methylbenzyl)-1,3,4-oxadiazolyl]carbonyl)-2-(S)-methylpropyl]acetamide; 1-Acetyl-3-[(4-pyridyl)methylene]piperazine-2,4-dione-N-[1-(2-(5-(3-methyl benzyl)-1,3,4-oxadiazolyl]carbonyl)-2-(S)-methylpropyl]acetamide; 4-[1-Benzyl-3-(R)-benzyl-piperazine-2,5,-dione]-N-[1-(2-[5-(3-methylbenzyl)-1,3,4-oxadiazolyl]carbonyl)-2-(S)-methylpropyl]acetamide; 4-[1-Benzyl-3-(S)-benzyl piperazine-2,5,-dione]-N-[1-(2-(5-(3-methylbenzyl)-1,3,4-oxadiazolyl]carbonyl)-2-(S)-methylpropyl]acetamide; 4-[1-Benzyl-3(R)-benzylpiperazine-2,5,-dione]-N-[1-(3-(5-(3-trifluoromethylbenzyl)-1,2,4-oxadiazolyl]carbonyl)-2-(S)-methylpropyl]acetamide; [B 4-[1-Benzyl-3-(S)-benzylpiperazine-2,5,-dione]-N-[1-(3-(5-(3-trifluoromethylbenzyl)-1,2,4-oxadiazolyl]carbonyl)-2-(S)-methylpropyl]acetamide; 4-[1-Benzyl-3-(S)-benzyl piperazine-2,5,-dione]-N-[1-(3-(5-(2-dimethylaminoethyl)-1,2,3--oxadiazolyl ]carbonyl)-2-(S)-methylpropyl]acetamide; 4-[1-Methyl-3-(R,S)-phenylpiperazine-2,5,-dione]-N-[1-(3-(5-(3-trifluoromethylbenzyl)-1,2,3--oxadiazolyl]carbonyl)-2-(S)-methylpropyl]acetamide; 4-[[-Methyl-3-(R,S)-phenyl piperazine-2,5,-dione]-N-[1-(2-(5-(3-methylbenzyl)-1,3,4-oxadiazolyl]carbonyl)-2-(S)-methylpropyl]acetamide; 4-[1-(4-Morpholino ethyl)3-(R)-benzyl piperazine-2,5,-dione]-N-[1-(2-(5-(3-methylbenzyl)-1,3,4-oxadiazolyl]carbonyl)-2-(S)-methylpropyl]acetamide; 5-(R,S)-Phenyl-2,4-imidazolidinedione-N-[1-(2-(5-(3-methylbenzyl)-1,3,4-oxadiazolyl]carbonyl)-2-(S)-methylpropyl]acetamide; 5-(R)-Benzyl-2,4-imidazolidinedione-N-[1-(2-(5-(3-methylbenzyl)-1,3,4-oxadiazolyl]carbonyl)-2-(S)-methylpropyl]acetamide; 5-(S)-Benzyl-2,4-imidazolidinedione-N-[1-(2-(5-(3-methylbenzyl)-1,3,4-oxadiazolyl]carbonyl)-2-(S)-methylpropyl]acetamide; 5-(S)-Benzyl-2,4-imidazolidinedione-N-[1-(3-(5-(3-trifluoromethylbenzyl)-1,2,4-oxadiazolyl]carbonyl)-2-(S)-methylpropyl]acetamide; 5-(R)-Benzyl-2,4-imidazolidinedione-N-[1-(3-(5-(3-trifluoromethylbenzyl)-1,2,4-oxadiazolyl]carbonyl)-2-(S)-methylpropyl]acetamide; 1-Benzyl-4-(R)-benzyl-2,4-imidazolidinedione-N-[1-(2-(5-(3-methylbenzyl)-1,3,4-oxadiazolyl]carbonyl)-2-(S)-methylpropyl]acetamide; and 1-Benzyl-4-(R)-benzyl-2,4-imidazolidinedione-N-[1-(3-(5-(3-trifluoromethyl benzyl)-1,2,4-oxadiazolyl]carbonyl)-2-(S)-methylpropyl]acetamide, or mixtures thereof.

16. A pharmaceutical composition for the treatment of a herpes virus infection, which comprises a peptide of the general formula: N_{T}—X₁—X₂—X₃—X₄—X₅—C_{T} or a physiologically acceptable salt thereof, in which N_{T} comprises an amino acid residue positioned at the peptide's N-terminal end, including C, an acetyl group, or a succinyl group, provided that N_{T} can also be absent; X₁ comprises an amino acid residue, including F or A; X₂ comprises an amino acid residue, including C, V, L, M, I, A, C, or S; X₃ comprises an amino acid residue, including F, A, V, M, L, I, Y, or C; X₄ comprises an amino acid residue, including L, A, F, I, V, M, C, G, or S; X₅ comprises an amino acid residue, including M, A, I, L, V, F, or G; and C_{T} comprises an amino acid residue positioned at the peptide's C-terminal end, including C, an amide group, a substituted amide group, or an ester group, provided that C_{T} can also be absent, and in which the amino acid residue can be either an L- or a D-stereoisomeric configuration.

17. A method of treating a mammal suffering from a herpes virus infection that is mediated by endogenous host serine protease (SP) or SP-like activity, which comprises administering to said mammal a therapeutically effective amount of a substance exhibiting mammalian alpha-1-antitrypsin (AAT) or AAT-like activity.

18. A method of inhibiting in a mammal the spread or onset of a viral infection that is mediated by endogenous host serine protease (SP) or SP-like activity, which comprises administering a therapeutically effective amount of a substance exhibiting mammalian alpha-1-antitrypsin (AAT) or AAT-like activity to a mammalian subject exposed or at risk of potential exposure to an agent of a viral infection that is mediated by endogenous host serine protease (SP) or SP-like activity.

19. A method of treating a patient with a deficiency of functional endogenous AAT levels and suffering from a viral infection that is mediated by endogenous host serine protease (SP) or SP-like activity, which comprises administering to such a patient a therapeutically effective amount of a substance exhibiting mammalian alpha-1-antitrypsin (AAT) or AAT-like activity.

20. A method of treating an individual suffering from a viral infection that is mediated at least in part by serine protease activity, which comprises administering to such an individual a therapeutically effective amount of a substance exhibiting mammalian alpha-1-antitrypsin (AAT) or AAT-like activity.

21. A method of preventing a deficiency of functional endogenous AAT levels in a mammalian patient susceptible to a viral infection that is mediated by endogenous host serine protease (SP) or SP-like activity, which comprises administering to such a mammalian patient a therapeutically effective amount of a substance exhibiting mammalian alpha-1-antitrypsin (AAT) or AAT-like activity.

22. The method of claim 21 in which the substance is (benzyloxycarbonyl)-L-valyl-N-[1-(3-(5-(3-trifluoromethylbenzyl)-1,2,4-oxadiazolyl)carbonyl)-2-(S)-methylpropyl]-L-prolinamide benzyloxycarbonyl)-L-valyl-N-[1-(3-(5-(2-phenylethyl)-1,2,4-oxadiazolyl)carbonyl)-2-(S)-methylpropyl]-L-prolinamide; (benzyloxycarbonyl)-L-valyl-N-[1-(3-(5-(2-methoxybenzyl)-1,2,4-oxadiazolyl)carbonyl)-2-(S)-methylpropyl]-L-prolinamide; (benzyloxycarbonyl)-L-valyl-N-[1-(3-(5-(trifluoromethyl)-1,2,4-oxadiazolyl)carbonyl)-2-(S)-methylpropyl]-L-prolinamide; (benzyloxycarbonyl)-L-valyl-N-[1-(3-(5-(methyl)-1,2,4-oxadiazolyl)carbonyl)-2-(S)-Methylpropyl]-L-Prolinamide; (Benzyloxycarbonyl)-L-Valyl-N-[1-(3-(5-(difluoromethyl)-1,2,4-oxadiazolyl) carbonyl)-2-(S)-Methylpropyl]-L-Prolinamide; (Benzyloxycarbonyl)-L-Valyl-N-[1-(3-(5-(benzyl)-1,2,4-oxadiazolyl)carbonyl)-2-(S)-Methylpropyl]-L-Prolinamide; (Benzyloxycarbonyl)-L-Valyl-N-[1-(3-(5-(3-methoxybenzyl)-1,2,4-oxadiazolyl)carbonyl)-2-(S)-methylpropyl]-L-Prolinamide; (Benzyloxycarbonyl)-L-Valyl-N-[1-(3-(5-(2,6-difluorobenzyl)-1,2,4-oxadiazolyl )carbonyl)-2-(S)-Methylpropyl]-L-Prolinamide; (Benzyloxycarbonyl)-L-Valyl-N-[1-(3-(5-(trans-styryl)-1,2,4-oxadiazolyl)carbonyl)-2-(S)-Methylpropyl]-L-Prolinamide; (Benzyloxycarbonyl)-L-Valyl-N-[1-(3-(5-(trans-4-Trifluoro methylstyryl)-1,2,4-oxadiazolyl)carbonyl)-2-(S)-Methylpropyl]-L-Prolinamide; (Benzyloxycarbonyl)-L-Valyl-N-[1-(3-(5-(trans-4--Methoxystyryl)-1,2,4-oxadiazolyl)carbonyl)-2-(S)-Methylpropyl]-L-Prolinamide; (Benzyloxycarbonyl)-L-Valyl-N-[1-(3-(5-(3-Thienylmethyl)-1,2,4-oxadiazolyl)carbonyl)-2-(S)-Methylpropyl]-L-Prolinamide; (Benzyloxycarbonyl)-L-Valyl-N-[1-(3-(5-(Phenyl)-1,2,4-oxadiazolyl)carbonyl )-2-(S)-methylpropyl]-L-prolinamide; and (Benzyloxycarbonyl)-L-Valyl-N-[1-(3-(5-(3-Phenylpropyl)-1,2,4-oxadiazolyl)carbonyl)-2-(S)-Methylpropyl]-L-Prolinamide, Benzyloxycarbonyl-L-valyl-N-[1-(2-[-5-(3-methylbenzyl)-1,3,4-oxadiazolyl]carbonyl)-2-(S)-methylpropyl]-L-prolinamide, Benzyloxycarbonyl-L-valyl-N-[1-(2-(3-methylbenzyl)-1,3,4-oxadiazolyl]carbonyl)-2-(S)-methylpropyl]-L-prolinamide; Benzyloxycarbonyl-L-valyl-N-[1-(2-(5-(methyl)-1,3,4-oxadiazoly]carbonyl)-2-(S)-methylpropyl]-L-prolinamide; Benzyloxycarbonyl)-L-valyl-N-[1-(2-(5-(3-trifluoromethylbenzyl)-1,3,4-oxadiazolyl]carbonyl)-2-(S)-methylpropyl]-L-prolinamide; (Benzyloxycarbonyl)-L-valyl-N-[1-(2-(5-(4-Dimethylamino benzyl)-1,2,4-oxadiazolyl]carbonyl)-2-(S)-methylpropyl]-L-prolinamide; Benzyloxycarbonyl)-L-valyl-N-[1-(2-(5-(1 -napthylenyl)-1,3,4-oxadiazolyl]carbonyl)-2-(S)-methylpropyl]-L-prolinamide; (Benzyloxycarbonyl)-L-valyl-[1-(3-(5-(3,4-methylenedioxybenzyl)-1,2,4-oxadiazolyl]carbonyl)-2-(S)-methylpropyl]-L-prolinamide; Benzyloxycarbonyl)-L-valyl-N-[1-(3-(5-(3,5-dimethylbenzyl)-1,2,4-oxadiazolyl]carbonyl)-2-(S)-methylpropyl]-L-prolinamide; (Benzyloxycarbonyl)-L-valyl-N-[1-(3-(5-(3,5-dimethoxybenzyl)-1,2,4-oxadiazolyl]carbonyl)-2-(S)-methylpropyl]-L-prolinamide; (Benzyloxycarbonyl)-L-valyl-N-[1-(3-(5-(3,5-ditrifluoromethylbenzyl)-1,2,4-oxadiazolyl]carbonyl)-2-(S)-methylpropyl]-L-prolinamide; (Benzyloxycarbonyl)-L-valyl-N-[1-(3-(5-(3-methylbenzyl)-1,2,4-oxadiazolyl]carbonyl)-2-(S)-methylpropyl]-L-prolinamide; (Benzyloxycarbonyl)-L-valyl-N-[1-(3-(5-(biphenylmethine)-1,2,4-oxadiazolyl]carbonyl)-2-(S)-methylpropyl]-L-prolinamide; (Benzyloxycarbonyl)-L-valyl-N-[1-(3-(5-(4-phenylbenzyl)-1,2,4-oxadiazolyl]carbonyl)-2-(S)-methylpropyl]-L-prolinamide; (Benzyloxycarbonyl)-L-valyl-N-[1-(3-(5-(3-phenylbenzyl)-1,2,4-oxadiazolyl]carbonyl)-2-(S)-methylpropyl]-L-prolinamide; (Benzyloxycarbonyl)-L-valyl-N-[1-(3-(5-(3-phenoxybenzyl)-1,2,4-oxadiazolyl ]carbonyl)-2-(S)-methylpropyl]-L-prolinamide; (Benzyloxycarbonyl)-L-valyl-N-[1-(3-(5-(cyclohexylmethylene)-1,2,4-oxadiazolyl]carbonyl)-2-(S)-methylpropyl]-L-prolinamide; (Benzyloxycarbonyl)-L-valyl-N-[1-(3-(5-(3-trifluoromethyldimethylmethylene )-1,2,4-oxadiazolyl]carbonyl)-2-(S)-methylpropyl]-L-prolinamide; (Benzyloxycarbonyl)-L-valyl-N-[1-(3-(5-(1-napthylmethylene)-1,2,4-oxadiazolyl]carbonyl)-2-(S)-methylpropyl]-L-prolinamide; (Benzyloxycarbonyl)-L-valyl-N-[1-(3-(5-(3-pyridylmethyl)-1,2,4-oxadiazolyl ]carbonyl)-2-(S)-methylpropyl]-L-prolinamide; (Benzyloxycarbonyl)-L-valyl-N-[1-(3-(5-(3,5-diphenylbenzyl)-1,2,4-oxadiazolyl]carbonyl)-2-(S)-methylpropyl]-L-prolinamide; (Benzyloxycarbonyl)-L-valyl-N-[1-(3-(5-(4-dimethylaminobenzyl)-1,2,4-oxadiazolyl]carbonyl)-2-(S)-methylpropyl]-L-prolinamide; 2-(5-[(Benzyloxycarbonyl)amino]-6-oxo-2-(4-fluorophenyl)-1,6-dihydro-1-pyrimidinyl]-N-[1-(3-(5-(3-trifluoromethylbenzyl)-1,2,4-oxadiazolyl]carbonyl)-(S)-2-methylpropyl]acetamide; 2-(5-Amino-6-oxo-2-(4-fluorophenyl)-1,6-dihydro-1-pyrimidinyl]-N-[1-(3-(5-(3-trifluoromethylbenzyl)-1,2,4-oxadiazolyl]carbonyl)-2-(S)-methylpropyl]acetamide; 2-(5-[(Benzyloxycarbonyl )amino]-6-oxo-2-(4-fluorophenyl)-1,6-dihydro-1-pyrimidinyl]-N-[1-(2-(5-(3-methylbenzyl)-1,3,4-oxadiazolyl]carbonyl)-(S)-2-methylpropyl]acetamide; 2-(5-Amino-6-oxo-2-(4-fluorophenyl)-1,6-dihydro-1-pyrimidinyl]-N-[1-(2-(5-(3-methylbenzyl)-1,3,4-oxadiazolyl]carbonyl)-2-methylpropyl]acetamide; (Pyrrole-2-carbonyl)-N-(benzyl)glycyl-N-[1-(2-(5-(3-methylbenzyl)-1,2,3-oxadiazolyl]carbonyl)-2-(S)-methylpropyl]amide; (Pyrrole-2-carbonyl)-N-(benzyl)glycyl-N-[1-(3-(5-(3-trifluoromethylbenzyl) ]-1,2,4-oxadiazolyl)-(S)-methylpropyl]amide; (2S,5S)-5-Amino-1,2,4,5,6,7-hexahydroazepino-[3,2,1]-indole-4-one-carbonyl -N-[1-(2-(5-(3-methylbenzyl)-1,3,4-oxadiazolyl]carbonyl)-(R,S)-2-methylpropyl]amide; BTD-[1-(2-(5-(3-methylbenzyl)-1,3,4-oxadiazolyl]carbonyl)-2-(S)-methylpropyl]amide; (R,S)-3-Amino-2-oxo-5-phenyl-1,4,-benzodiazepine-N-[1-(2-(5-(3-methylbenzyl)-1,3,4-oxadiazolyl]carbonyl)-2-(S)-methylpropyl]acetamide; (Benzyloxycarbonyl)-L-valyl-2-L-(2,3-dihydro-1H-indole)-N-[1-(2-(5-(3 -methylbenzyl)-1,3,4-oxadiazolyl]carbonyl)-2-(S)-methylpropyl]amide; (Benzyloxycarbonyl)-L-valyl-2-L-(2,3-dihydro-1H-indole)-N-[1-(3-(5-(3-trifluoromethylbenzyl)-1,2,4-oxadiazolyl]carbonyl)-2-(S)-methylpropyl]amide; Acetyl-2-L-(2,3-dihydro-1H-indole)-N-[1-(2-(5-(3-methylbenzyl)-1,3,4-oxadiazolyl]carbonyl)-2-(S)-methylpropyl]amide; 3-(S)-(Benzyloxycarbonyl)amino)-εe-lactam-N-[1-(2-(5-(3-methylbenzyl)-1,3, 4-oxadiazolyl]carbonyl )-2-(S)-methylpropyl]acetamide; 3-(S)-(Amino)-ε-lactam-N-[1-(2-(5-(3-methylbenzyl)-1,3,4-oxadiazolyl]carbonyl)-2-(S)-methylpropyl]acetamide trifluoroacetic acid salt; 3-(S)-[(4-morpholino carbonyl-butanoyl)amino]-ε-lactam-N-[1-(2-(5-(3-methylbenzyl)-1,3,4-oxadiazolyl]carbonyl)-2-(R,S)-methylpropyl]acetamide; 6-[4-Fluorophenyl]-ε-lactam-N-[1-(2-(5-(3-methylbenzyl)-1,3,4-oxadiazolyl]carbonyl)-2-(S)-methylpropyl]acetamide; 2-(2-(R,S)-Phenyl-[4-oxothiazolidin-3-yl]-N-[1-(2-(5-(3-methylbenzyl)-1,3,4-oxadiazolyl]carbonyl)-2-(S)-methylpropyl]acetamide; 2-(2-(R,S)-phenyl-4-oxothiazolidin-3-yl]-N-[1-(2-(5-(3-methylbenzyl)-1,3,4-oxadiazolyl]hydroxymethyl)-2-(S )-methylpropyl]acetamide; 2-(2-(R,S)-Benzyl-4-oxothiazolidin-3-yl]-N-[1-(2-(5-(3-methylbenzyl)-1,3,4-oxadiazolyl]carbonyl)-2-(S)-methylpropyl]-acetamide; 2-(2-(R,S)-Benzyl-4-oxothiazolidin-3-yl oxide]-N-[1-(3-(5-(3-trifluoromethylbenzyl)-1,2,4-oxadiazolyl]carbonyl)-2-(R,S,)-methylpropyl]acetamide; (1-Benzoyl-3,8-quinazolinedione)-N-[1-(2-(5-(3-methylbenzyl)-1,3,4-oxadiazolyl]carbonyl)-2-(S)-methylpropyl]acetamide; (1-Benzoyl-3,6-piperazinedione)-N-[1-(2-(5-(3-methylbenzyl)-1,3,4-oxadiazolyl]carbonyl)-2-(S)-methylpropyl]acetamide; (1-Phenyl-3,6-piperazinedione)-N-[1-(2-(5-(3-methylbenzyl)-1,3,4-oxadiazolyl]carbonyl)-2-(S)-methylpropyl]acetamide; [(1-Phenyl-3,6-piperazinedione)N-[3-(5-3-trifluoromethylbenzyl)-1,2,4-oxadiazolyl]carbonyl)]-2-(S)-methylpropyl]acetamide; 3-[(Benzyloxycarbonyl)amino]-quinolin-2-one-N-[1-(2-(5-(3-methylbenzyl)-1,3,4-oxadiazolyl]carbonyl)-2-(S)-methylpropyl]acetamide; 3-[(Benzyloxycarbonyl)amino]-7-piperidinyl-quinolin-2-one-N-[1-(2-(5-(3-methylbenzyl)-1,3,4-oxadiazolyl]carbonyl)-2-(S)-methylpropyl]acetamide; 3-(Carbomethoxy-quinolin-2-one-N-[1-(2-(5-(3-methybenzyl)-1,3,4-oxadiazolyl]carbonyl)-2-(S)-methylpropyl]acetamide; 3-(Amino-quinolin-2-one)-N-[1-(2-(5-(3-methylbenzyl)-1,3,4-oxadiazolyl]carbonyl)-2-(S)-methylpropyl]acetamide; 3-[(4-Morpholino)aceto]amino-quinolin-2-one-N-[1-(2-(5-(3-methylbenzyl)-1,3,4-oxadiazolyl]carbonyl)-2-(S)-methylpropyl]acetamide; 3,4-Dihydro-quinolin-2-one-N-[1-(2-(5-(3-methylbenzyl)-1,3,4-oxadiazolyl ]carbonyl)-2-(S)-methylpropyl]acetamide; 1-Acetyl-3-(4-fluorobenzylidene) piperazine-2,5-dione-N-[1-(2-(5-(3-methylbenzyl)-1,3,4-oxadiazolyl]carbonyl)-2-(S)-methylpropyl]acetamide; 1-Acetyl-3-(4-dimethylamino benzylidene)piperazine-2,5-dione-N-[1-(2-(5-(3-methylbenzyl)-1,3,4-oxadiazolyl]carbonyl)-2-(S)-methylpropyl]acetamide; 1-Acetyl-3-(4-carbomethoxy benzylidene)piperazine-2,5-dione-N-[1-(2-(5-(3-methylbenzyl)-1,3,4-oxadiazolyl]carbonyl)-2-(S)-methylpropyl]acetamide; 1-Acetyl-3-[(4-pyridyl)methylene]piperazine-2,5-dione-N-[1-(2-(5-(3-methylbenzyl)-1,3,4-oxadiazolyl]carbonyl)-2-(S)-methylpropyl]acetamide; 4-[1-Benzyl-3-(R)-benzyl-piperazine-2,5,-dione]-N-[1-(2 -[-5-(3-methylbenzyl)-1,3,4-oxadiazolyl]carbonyl)-2-(S)-methylpropyl]acetamide; 4-[1-Benzyl-3-(S)-benzylpiperazine-2,5,-dione]-N-[1-(2-(5-(3-methylbenzyl)-1,3,4-oxadiazolyl]carbonyl)-2-(S)-methylpropyl]acetamide; 4-[1-Benzyl-3(R)-benzylpiperazine-2,5,-dione]-N-[1-(3-(5-(3-trifluoromethylbenzyl)-1,2,4-oxadiazolyl]carbonyl)-2-(S)-methylpropyl]acetamide; 4-[1-Benzyl-3-(S)-benzylpiperazine-2,5,-dione]-N-[1-(3-(5-(3-trifluoromethylbenzyl)-1,2,4-oxadiazolyl]carbonyl)-2-(S)-methylpropyl]acetamide; 4-[1-Benzyl-3-(S)-benzylpiperazine-2,5,-dione]-N-[1-(3-(5-(2-dimethylaminoethyl)-1,2,4-oxadiazolyl ]carbonyl)-2-(S)-methylpropyl]acetamide; 4-[1-Methyl-3-(R,S)-phenylpiperazine-2,5,-dione]-N-[1-(3-(5-(3-trifluoromethylbenzyl)-1,2,4-oxadiazolyl]carbonyl)-2-(S)-methylpropyl]acetamide; 4-[[-Methyl-3-(R,S)-phenylpiperazine-2,5,-dione]-N-[1-(2-(5-(3-methylbenzyl)-1,3,4-oxadiazolyl]carbonyl)-2-(S)-methylpropyl]acetamide; 4-[1-(4-Morpholinoethyl)3-(R)-benzylpiperazine-2,5,-dione]-N-[1-(2-(5-(3-methylbenzyl)-1,3,4-oxadiazolyl]carbonyl)-2-(S)-methylpropyl]acetamide; 5-(R,S )-Phenyl-2,4-imidazolidinedione-N-[1-(2-(5-(3-methylbenzyl)-1,3,4-oxadiazolyl]carbonyl)-2-(S)-methylpropyl ]acetamide; 5-(R)-Benzyl-2,4-imidazolidinedione-N-[1-(2-(5-(3-methylbenzyl)-1,3,4-oxadiazolyl]carbonyl)-2-(S)-methylpropyl]acetamide; 5-(S)-Benzyl-2,4-imidazolidinedione-N-[1-(2-(5-(3-methylbenzyl)-1,3,4-oxadiazolyl]carbonyl)-2-(S)-methylpropyl ]acetamide; 5-(S)-Benzyl-2,4-imidazolidinedione-N-[1-(3-(5-(3-trifluoromethylbenzyl)-1,2,4-oxadiazolyl]carbonyl)-2-(S)-methylpropyl]acetamide; 5-(R)-Benzyl-2,4-imidazolidinedione-N-[1-(3-(5-(3-trifluoromethylbenzyl)-1,2,4-oxadiazolyl]carbonyl)-2-(S)-methylpropyl]acetamide; 1-Benzyl-4-(R)-benzyl-2,5-imidazolidinedione-N-[1-(2-(5-(3-methylbenzyl)-1,3,4-oxadiazolyl]carbonyl)-2-(S)-methylpropyl]acetamide; and 1-Benzyl-4-(R)-benzyl-2,5-imidazolidinedione-N-[1-(3-(5-(3-trifluoromethylbenzyl)-1,2,4-oxadiazolyl]carbonyl)-2-(S)-methylpropyl]acetamide, or mixtures thereof.

23. A pharmaceutical composition comprising effective amounts of a substance exhibiting mammalian alpha-1-antitrypsin (AAT) or AAT-like activity and a 15 pharmaceutically acceptable carrier.

24. The pharmaceutical composition of claim 23 in which the substance comprises AAT.

25. The pharmaceutical composition of claim 23 in which the substance comprises a peptide or a synthetic serpin, which exhibits AAT or AAT-like activity.

26. A method for the treatment of pre-existing lesions and sores of the skin or mucosa associated with a herpes virus and for prevention of future lesions and sores of the skin or mucosa associated with a herpes virus, comprising administering an effective amount of a topical preparation comprising a substance exhibiting mammalian AAT- or AAT-like activity.

27. The method of claim 26 in which said topical preparation further comprises a pharmaceutically or cosmetically acceptable carrier.

28. A method for treating or preventing herpes, comprising administering to a patient in need thereof an effective amount of a substance exhibiting AAT- or AAT-like activity.

29. The method of claim 28 in which said substance comprises AAT administered in a unit dose form having from about 50 to about 1000 mg of AAT.

30. The method of claim 28 in which said substance comprises a peptide selected from FVFLM (SEQ. ID NO. 1), FVFAM (SEQ. ID NO. 2), FVALM (SEQ. ID NO. 3), FVFLA (SEQ. ID NO. 4), FLVFI (SEQ. ID NO. 5), FLMII (SEQ. ID NO. 6), FLFVL (SEQ. ID NO. 7), FLFVV (SEQ. ID NO. 8), FLFLI (SEQ. ID NO. 9), FLFFI (SEQ. ID NO. 10), FLMFI (SEQ. ID NO. 11), FMLLI (SEQ. ID NO. 12), FIIMI (SEQ. ID NO. 13), FLFCI (SEQ. ID NO. 14), FLFAV (SEQ. ID NO. 15), FVYLI (SEQ. ID NO. 16), FAFLM (SEQ. ID NO. 17), AVFLM (SEQ. ID NO. 18), FCICV (SEQ. ID NO. 19), FCVCF (SEQ. ID NO. 20), FIVCV (SEQ. ID NO. 21), FCVGV (SEQ. ID NO. 22), FCVLV (SEQ. ID NO. 23), FLVGV (SEQ. ID NO. 24), FSVSV (SEQ. ID NO. 25), FSVCV (SEQ. ID NO. 26), FVCVG (SEQ. ID NO. 27), or combinations thereof.

31. A method of preventing sexually transmitted diseases comprising administering intravaginally or intrarectally an effective amount of a substance having AAT- or AAT-like activity or a derivative thereof capable of inhibiting caspase, proteinase-3, cathepsin G, elastase, or combinations thereof.

32. The method of claim 31 in which the sexually transmitted viral disease is caused by a herpes virus.

33. A method of treating a herpes virus infection in an animal subject comprising topically administering to the subject an effective amount of one or more compounds with AAT- or AAT-like activity.

34. The method of claim 33 in which the said one or more compounds is present in a preparation further comprising a pharmaceutically or cosmetically acceptable carrier.

35. The method of claim 34 in which said preparation is in the form of a solution, lotion, ointment, or cream.

36. The method of claim 33 in which the herpes virus infection is caused by herpes virus type 1 or 2, varicella zoster virus, CMV, EBV, HHV-5, HHV-6, HHV-8, or combinations thereof.

37. The method of claim 33 which further comprises administering to the subject an effective amount of a second compound selected from anesthetics, analgesics, antibiotics, or combinations thereof.

38. A method of preventing or inhibiting entry of herpes viral nucleic acid into a mammalian host cell nucleus, which comprises administering to a mammalian host exposed or at risk of potential exposure to an agent harboring herpes viral nucleic acid an effective amount of a substance exhibiting mammalian alpha-1-antitrypsin (AAT) or AAT-like activity.

39. The method of claim 38 in which the entry of said herpes viral nucleic acid is mediated by endogenous host serine protease (SP) or SP-like activity.
